SEQUENCE LISTING (32 sequences)

SEQ ID NO: 1 - protein, 134 aa, Escherichia coli
MNTTLFRWPVRVYYEDTDAGGVVYHASYVAFYERARTEMLRHHHFSQQALMAERVAFVVRKMTVEYYAPARLDDMLEIQTEITSMRGTSLVFTQRIVNAENTLLNEAEVLVVCVDPLKMKPRALPKSIVAEFKQ

SEQ ID NO: 2 - protein, 132 aa, Citrobacter koseri
MNIFRWPVRVYYEDTDAGGVVYHASYVAFYERARTEMLRHHHFSQQVLLAERVAFVVRKMTLEYYAPARLDDMLEVQTEITSMRGTSLVFTQRIVNAENNVLNEAEVLIVCVDPLKMKPRALPKSIVAEFKQ

SEQ ID NO: 3 - protein, 134 aa, Enterobacter cloacae
MNTTLFRWPVRVYYEDTDAGGVVYHASYVAFYERARTEMLRHHHFSQQVLLAERVAFVVRKMTLEYFAPARLDDMLEIQTEITSMRGTSLVFTQRIVNAENTVLNEAEVLIVCVDPLLMKPRALPKSIVAEFKQ

SEQ ID NO: 4 - protein, 135 aa, Serratia fonticola
MSNKLFRWPVRVYFEDTDAGGVVYHARYVAFYERARTEMLRQHNFHQQQLLSEEHVAFAVRRMTVDYLSPARLDDLLEVQSKITSIRGASLTFAQRIVNSDGTLLSQADVLIACIDPHQMKPIALPKSIVAEFKQ

SEQ ID NO: 5 - protein, 124 aa, Exiguobacterium mexicanum
MLFRWPVRVYYEDTDAGGVVYHARYLAFFERARTEMLRNKGINQQSMLADNLGFVVRSMTIDFVKGAKLDDLLEVETQIVEMKRASLTFHQRLVDSQGNLLCGANALIACVDTSKMKPIALPKS

SEQ ID NO: 6 - protein, 136 aa, Plesiomonas shigelloides
MSSKVSQWPVRVYYEDTDAGGVVYHARYVAFFERARTEMLRSLGQQQQTLLQHNVAFVVRRMTVDYRSPARLDDLLSVETEISSFRGASVTFNQRIIHQDGRVLCTAEVLVACIDLAKMKPVALPAELVAEFTCVC

SEQ ID NO: 7 - protein, 560 aa, Escherichia coli
MKKVWLNRYPADVPTEINPDRYQSLVDMFEQSVARYADQPAFVNMGEVMTFRKLEERSRAFAAYLQQGLGLKKGDRVALMMPNLLQYPVALFGILRAGMIVVNVNPLYTPRELEHQLNDSGASAIVIVSNFAHTLEKVVDKTAVQHVILTRMGDQLSTAKGTVVNFVVKYIKRLVPKYHLPDAISFRSALHNGYRMQYVKPELVPEDLAFLQYTGGTTGVAKGAMLTHRNMLANLEQVNATYGPLLHPGKELVVTALPLYHIFALTINCLLFIELGGQNLLITNPRDIPGLVKELAKYPFTAITGVNTLFNALLNNEFQQLDFSSLHLSAGGGMPVQQVVAERWVKLTGQYLLEGYGLTECAPLVSVNPYDIDYHSGSIGLPVPSTEAKLVDDDDNEVPPGQPGELCVKGPQVMLGYWQRPDATDEIIKNGWLHTGDIAVMDEEGFLRIVDRKKDMILVSGFNVYPNEIEDVVMQHPGVQEVAAVGVPSGSSGEAVKIFVVKKDPSLTEESLVTFCRRQLTGYKVPKLVEFRDELPKSNVGKILRRELRDEARGKVDNKA

SEQ ID NO: 8 - protein, 565 aa, Pseudomonas putida
MIENFWKDKYPAGITAEINPDEFPNIQAVLKQSCQRFADKPAFSNLGKTITYGELYALSGAFAAWLQQHTDLKPGDRIAVQLPNVLQYPVAVFGAMRAGLIVVNTNPLYTAREMEHQFNDSGAKALVCLANMAHLAEKVVPKTQVRHVIVTEVADLLPPLKRLLINSVIKYVKKMVPAYNLPQAVRFNDALALGKGQPVTEANPQANDVAVLQYTGGTTGVAKGAMLTHRNLVANMLQCRALMGSNLHEGCEILITPLPLYHIYAFTFHCMAMMLIGNHNVLISNPRDLPAMVKELGKWKFSGFVGLNTLFVALCNNEAFRALDFSALKITLSGGMALQLSVAERWKAVTGCAICEGYGMTETSPVAAVNPSEANQVGTIGIPVPSTLCKVIDDAGNELPLGEVGELCVKGPQVMKGYWQREDATAEILDSEGWLKTGDIAVIQADGYMRIVDRKKDMILVSGFNVYPNELEDVLAALPGVLQCAAIGVPDEKSGEVIKVFIVVKPGMTVTKEQVMEHMRANVTGYKVPRHIEFRDSLPTTNVGKILRRELRDEELKKQGLKKIA

SEQ ID NO: 9 - protein, 562 aa, Pseudomonas putida
MQADFWNDKRPAGVPSTIDINAYASVVEVFERSCKRFADRPAFSNLGVTLSYAELERHSAAFAAWLQQHTDLKPGERIAVQMPNVLQYPIAVFGAMRAGLIVVNTNPLYTEREMRHQFKDSGARALVYLNMFGKRVQEVLPDTGIEYLIEAKMGDLLPAAKGWLVNTVVDKLKKMVPAYRLPQAVPFKQVLREGRGLSPKPVSLNLDDIAVLQYTGGTTGLAKGAMLTHGNLVANMLQVLACFSQHGPDGQKLLKDGQEVMIAPLPLYHIYAFTANCMCMMVTGNHNVLITNPRDIPGFIKELGKWRFSALLGLNTLFVALMDHPGFRQLDFSALKVTNSGGTALVKATAERWEDLTGCRIVEGYGLTETSPVASTNPYGQLARLGTVGIPVAGTAFKVIDDDGNELPLGERGELCIKGPQVMKGYWQQPEATAQALDAEGWFKTGDIAVIDPDGFTRIVDRKKDMIIVSGFNVYPNEIEDVVMGHPKVANCAAIGVPDERSGEAVKLFVVPREGGLSVDELKAYCKANFTGYKVPKHIVLRESLPMTPVGKILRRELRDIA

SEQ ID NO: 10 - protein, 560 aa, Pseudomonas putida
MLQTRIIKPAEGAYAYPLLIKRLLMSGSRYEKTREIVYRDQMRLTYPQLNERIARLANVLTEAGVKAGDTVAVMDWDSHRYLECMFAIPMIGAVVHTINVRLSPEQILYTMNHAEDRVVLVNSDFVGLYQAIAGQLTTVDKTLLLTDGPDKTAELPGLVGEYEQLLAAASPRYDFPDFDENSVATTFYTTGTTGNPKGVYFSHRQLVLHTLAEASVTGSIDSVRLLGSNDVYMPITPMFHVHAWGIPYAATMLGMKQVYPGRYEPDMLVKLWREEKVTFSHCVPTILQMLLNCPNAQGQDFGGWKIIIGGSSLNRSLYQAALARGIQLTAAYGMSETCPLISAAHLNDELQAGSEDERVTYRIKAGVPVPLVEAAIVDGEGNFLPADGETQGELVLRAPWLTMGYFKEPEKSEELWQGGWLHTGDVATLDGMGYIDIRDRIKDVIKTGGEWVSSLDLEDLISRHPAVREVAVVGVADPQWGERPFALLVARDGHDIDAKALKEHLKPFVEQGHINKWAIPSQIALVTEIPKTSVGKLDKKRIRQDIVQWQASNSAFLSTL

SEQ ID NO: 11 - protein, 560 aa, Pseudomonas aeruginosa
MLKTRLIPAAAGAYQYPLLIKSLMLSGRRYEKSHEIVYRDQVRYSYATFNERVARLANVLSEAGVKAGDTVAVMDWDSHRYLECMFAIPMIGAVLHTINIRLSPEQILYTMNHAEDRFVLVNSEFVPLYQAVAGQLATVERTILLTDGAEKSAELPGLVGEYESLLAAASPRYDFPDFDENSIATTFYTTGTTGNPKGVYFSHRQLVLHTLAMASTIGSLDSIRLLGTSDVYMPITPMFHVHAWGTPYVATMLGVKQVYPGRYDPELLVELWKREKVTFSHCVPTILQMVMNARAAQGVDFKGWKVIIGGSALNRSLYEAAKARGIQLTAAYGMSETCPLISCAYLNDELLAGSEDERTTYRIKAGVPVPLVDAAIMDEQGRFLPADGESQGELVLRSPWLTQGYFREPERGEELWRGGWMHTGDVATLDGMGFIEIRDRIKDVIKTGGEWLSSLELEDLISRHPAVREVAVVGVPDPQWGERPFALLVVREGQQLDARGLKEHLKPFVEQGNINKWAIPSQIAVVTDIPKTSVGKLDKKRIRIEIAQWQEAGSAFLSTV

SEQ ID NO: 12 - protein, 560 aa, Pseudomonas citronellolis
MLKTRLLPAASNAYQYPLLIKSLLLSGARYEKSREIVYRDLVRYNYATFNERVARLANVLTEAGVQAGDTVAVMDWDSHRYLEAMFAIPMIGAVLHTINIRLSPDQILYTMNHAEDRFVLVNAEFVPLYQGIAGQLTTVQKTLLLSDGADKSCALDDCIGEYETLMAAASPRYAFPDFDENSVATTFYTTGTTGNPKGVYFTHRQLVLHTLAMASTIASLDSIRLMGNSDVYMPITPMFHVHAWGVPYVATMLGIKQVYPGRYDPELLVDLWKRENVTFSHCVPTILQMVMNAKAAQGVDFKDWKIIIGGSALNRSLYEAAQARGIQLTAAYGMSETCPLISCAYLNDELLAGSEDERTTYRIKAGVPVPLVDAAIMDEHGQLLPADGESQGELVLRSPWLTQGYFREPQKGEELWAGGWMHTGDVATLDGMGFIDIRDRIKDVIKTGGEWLSSLELEDLISRHPAVREVAVVGVPDPQWGERPFALLVLFEGQQLDAKGLKEHLKPFVEQGHINKWAIPSQIAVVTEVPKTSVGKLDKKRIRVDIARWQESGSEFLSAL

SEQ ID NO: 13 - protein, 560 aa, Pseudomonas mendocina
MLQTRLIPPANNAHAYPLLIKRLLLSGSRYEKTREIVYRDKLRYSYATFTERVARLANVLSQAGVKAGDTVAVMDWDSHRYLECMFAIPMLGAVLHTINIRLSPDQILYTMNHAEDRFVLVNSEFVPLYNGIAGQLTTVEKTLLLTDGEDKNADLPNLVGEYESLLAAASPHYDFADFDENSVATTFYTTGTTGNPKGVYFTHRQLVLHTMSMATTMGGLDSIRLMGNDDVYMPITPMFHVHAWGVPYVATMLGVKQVYPGRYEPDMLCRLIKEEKVTFSHCVPTILQMLLSAPGAQGHDFGGMKMIIGGSALNRSLYEAAKERGIQLTAAYGMSETCPLISCAYLNEELRAGSEDERTTYRIKAGIPVPLVEAAIMDADGKLLPSDGESQGELVLRAPWLTQGYFREPEKGEELWAHGWLHTGDVATIDGMGFIEIRDRIKDVIKTGGEWISSLELEDLISRHSAVREVAVVGVPDPQWGERPFALVVLRDAQGLDAKGLKEHLKPFVEQGHINKWAIPTQIALVTEIPKTSVGKLDKKRIRVEIAQWQEAGSTFLSTL

SEQ ID NO: 14 - protein, 512 aa, Bacillus subtilis
MNLVSKLEETASEKPDSIACRFKDHMMTYQELNEYIQRFADGLQEAGMEKGDHLALLLGNSPDFIIAFFGALKAGIVVVPINPLYTPTEIGYMLTNGDVKAIVGVSQLLPLYESMHESLPKVELVILCQTGEAEPEAADPEVRMKMTTFAKILRPTSAAKQNQEPVPDDTAVILYTSGTTGKPKGAMLTHQNLYSNANDVAGYLGMDERDNVVCALPMFHVFCLTVCMNAPLMSGATVLIEPQFSPASVFKLVKQQQATIFAGVPTMYNYLFQHENGKKDDFSSIRLCISGGASMPVALLTAFEEKFGVTILEGYGLSEASPVTCFNPFDRGRKPGSIGTSILHVENKVVDPLGRELPAHQVGELIVKGPNVMKGYYKMPMETEHALKDGWLYTGDLARRDEDGYFYIVDRKKDMIIVGGYNVYPREVEEVLYSHPDVKEAVVIGVPDPQSGEAVKGYVVPKRSGVTEEDIMQHCEHLAKYKRPAAITFLDDIPKNATGKMLRRALRDILPQ

SEQ ID NO: 15 - protein, 700 aa, Saccharomyces cerevisiae
MVAQYTVPVGKAANEHETAPRRNYQCREKPLVRPPNTKCSTVYEFVLECFQKNKNSNAMGWRDVKEIHEESKSVMKKVDGKETSVEKKWMYYELSHYHYNSFDQLTDIMHEIGRGLVKIGLKPNDDDKLHLYAATSHKWMKMFLGAQSQGIPVVTAYDTLGEKGLIHSLVQTGSKAIFTDNSLLPSLIKPVQAAQDVKYIIHFDSISSEDRRQSGKIYQSAHDAINRIKEVRPDIKTFSFDDILKLGKESCNEIDVHPPGKDDLCCIMYTSGSTGEPKGVVLKHSNVVAGVGGASLNVLKFVGNTDRVICFLPLAHIFELVFELLSFYWGACIGYATVKTLTSSSVRNCQGDLQEFKPTIMVGVAAVWETVRKGILNQIDNLPFLTKKIFWTAYNTKLNMQRLHIPGGGALGNLVFKKIRTATGGQLRYLLNGGSPISRDAQEFITNLICPMLIGYGLTETCASTTILDPANFELGVAGDLTGCVTVKLVDVEELGYFAKNNQGEVWITGANVTPEYYKNEEETSQALTSDGWFKTGDIGEWEANGHLKIIDRKKNLVKTMNGEYIALEKLESVYRSNEYVANICVYADQSKTKPVGIIVPNHAPLTKLAKKLGIMEQKDSSINIENYLEDAKLIKAVYSDLLKTGKDQGLVGIELLAGIVFFDGEWTPQNGFVTSAQKLKRKDILNAVKDKVDAVYSSS

SEQ ID NO: 16 - protein, 362 aa, Umbellularia californica
MLKLSCNVTDSKLQRSLLFFSHSYRSDPVNFIRRRIVSCSQTKKTGLVPLRAVVSADQGSVVQGLATLADQLRLGSLTEDGLSYKEKFVVRSYEVGSNKTATVETIANLLQEVGCNHAQSVGFSTDGFATTTTMRKLHLIWVTARMHIEIYKYPAWGDVVEIETWCQSEGRIGTRRDWILKDSVTGEVTGRATSKWVMMNQDTRRLQKVSDDVRDEYLVFCPQEPRLAFPEENNRSLKKIPKLEDPAQYSMIGLKPRRADLDMNQHVNNVTYIGWVLESIPQEIVDTHELQVITLDYRRECQQDDVVDSLTTTTSEIGGTNGSATSGTQGHNDSQFLHLLRLSGDGQEINRGTTLWRKKPSS

SEQ ID NO: 17 - protein, 382 aa, Cinnamomum camphora
MATTSLASAFCSMKAVMLARDGRGMKPRSSDLQLRAGNAQTSLKMINGTKFSYTESLKKLPDWSMLFAVITTIFSAAEKQWTNLEWKPKPNPPQLLDDHFGPHGLVFRRTFAIRSYEVGPDRSTSIVAVMNHLQEAALNHAKSVGILGDGFGTTLEMSKRDLIWVVKRTHVAVERYPAWGDTVEVECWVGASGNNGRRHDFLVRDCKTGEILTRCTSLSVMMNTRTRRLSKIPEEVRGEIGPAFIDNVAVKDEEIKKPQKLNDSTADYIQGGLTPRWNDLDINQHVNNIKYVDWILETVPDSIFESHHISSFTIEYRRECTMDSVLQSLTTVSGGSSEAGLVCEHLLQLEGGSEVLRAKTEWRPKLTDSFRGISVIPAESSV

SEQ ID NO: 18 - protein, 419 aa, Ricinus communis
MVATAAAATSSFFPVPSQSADANFDKAPASLGGIKLKSTSCSRGLQVKANAQAPPKINGSSVGFTTSVETVKNDGDMPLPPPPRTFINQLPDWSMLLAAITTIFLAAEKQWMMLDWKPRRPDMLIDPFGIGRIVQDGLIFRQNFSIRSYEIGADRTASIETLMNHLQETALNHVKTAGLLGDGFGSTPEMSKRNLIWVVTRMQVLVDRYPTWGDVVQVDTWVSKSGKNGMRRDWCVRDSRTGETLTRASSVWVMMNKLTRRLSKIPEEVRGEIEPYFLNSDPIVDEDSRKLPKLDDSNADYVRKGLTPRWSDLDINQHVNNVKYIGWILESAPLPILESHELSAITLEYRRECGRDSVLQSLTAVSGNGIGNLGNAGDIECQHLLRLEDGAEIVRGRTEWRPKYSSNFGIMGQIPVESA

SEQ ID NO: 19 - protein, 415 aa, Cuphea hookeriana
MVAAAASSAFFPVPAPGASPKPGKFGNWPSSLSPSFKPKSIPNGGFQVKANDSAHPKANGSAVSLKSGSLNTQEDTSSSPPPRTFLHQLPDWSRLLTAITTVFVKSKRPDMHDRKSKRPDMLVDSFGLESTVQDGLVFRQSFSIRSYEIGTDRTASIETLMNHLQETSLNHCKSTGILLDGFGRTLEMCKRDLIWVVIKMQIKVNRYPAWGDTVEINTRFSRLGKIGMGRDWLISDCNTGEILVRATSAYAMMNQKTRRLSKLPYEVHQEIVPLFVDSPVIEDSDLKVHKFKVKTGDSIQKGLTPGWNDLDVNQHVSNVKYIGWILESMPTEVLETQELCSLALEYRRECGRDSVLESVTAMDPSKVGVRSQYQHLLRLEDGTAIVNGATEWRPKNAGANGAISTGKTSNGNSVS

SEQ ID NO: 20 - protein, 411 aa, Cuphea palustris
MVAAAASAAFFSVATPRTNISPSSLSVPFKPKSNHNGGFQVKANASAHPKANGSAVSLKSGSLETQEDKTSSSSPPPRTFINQLPVWSMLLSAVTTVFGVAEKQWPMLDRKSKRPDMLVEPLGVDRIVYDGVSFRQSFSIRSYEIGADRTASIETLMNMFQETSLNHCKIIGLLNDGFGRTPEMCKRDLIWVVTKMQIEVNRYPTWGDTIEVNTWVSASGKHGMGRDWLISDCHTGEILIRATSVWAMMNQKTRRLSKIPYEVRQEIEPQFVDSAPVIVDDRKFHKLDLKTGDSICNGLTPRWTDLDVNQHVNNVKYIGWILQSVPTEVFETQELCGLTLEYRRECGRDSVLESVTAMDPSKEGDRSLYQHLLRLEDGADIVKGRTEWRPKNAGAKGAILTGKTSNGNSIS

SEQ ID NO: 21 - protein, 419 aa, Cuphea lanceolata
MVAAAATSAFFPVPAPGTSPKPGKSGNWPSSLSPTFKPKSIPNAGFQVKANASAHPKANGSAVNLKSGSLNTQEDTSSSPPPRAFLNQLPDWSMLLTAITTVFVAAEKQWTMLDRKSKRPDMLVDSVGLKSIVRDGLVSRQSFLIRSYEIGADRTASIETLMNHLQETSINHCKSLGLLNDGFGRTPGMCKNDLIWVLTKMQIMVNRYPTWGDTVEINTWFSQSGKIGMASDWLISDCNTGEILIRATSVWAMMNQKTRRFSRLPYEVRQELTPHFVDSPHVIEDNDQKLHKFDVKTGDSIRKGLTPRWNDLDVNQHVSNVKYIGWILESMPIEVLETQELCSLTVEYRRECGMDSVLESVTAVDPSENGGRSQYKHLLRLEDGTDIVKSRTEWRPKNAGTNGAISTSTAKTSNGNSAS

SEQ ID NO: 22 - protein, 362 aa, Arabidopsis thaliana; identical to SEQ ID NO: 16

SEQ ID NO: 23 - protein, 365 aa, Helianthus annuus
MLSRGVPTATATAYNDVIKETARSVTNSRSIDSVSIRRRNNGFVSNSLCRRVAPVMAVKVDEQRTGVAVDVTEKRLADRLRMGSLTEDGLSYKERFIIRCYEVGINKTATVETIANLLQEVGGNHAQSVGFSTDGFATTTTMRKLNLIWVTSRMHIEIYRYPAWSDVVEIETWCQGEGRIGTRRDWIIKDHANGEVIGRATSKWVMMNSETRRLQKVNDDIRDEYLIFCPKTLRLAFPEENNNSLKKIAKLEDPAECSTLGLVPRRADLDMNKHVNNVTYIGWVLESIPQEVIDTHELQTITLDYRRECQHDDVVDSLTSSESPAVNGCASGENLSKFLHLLRSSGEGLELNRGRTEWRKKPAKK

SEQ ID NO: 24 - protein, 381 aa, Macadamia tetraphylla
MLSVKGCNAPDQLRSLTQCLSVTPPKALTRRGIAVVSCCSSSSTSPSSISASRVSNLVVSERTNGSGGVVSNPSSNGSASLLEELRLGSLAEDGLSYKEKFIVRSYEVGINKTATMEAIANLLQEAACNQVQTLGYSTDGMGTSYTMRQLHLIWVTARMHIEVYKYPAWGDMIEIETWFEAGRVGTRRDFVVKDATGTVIGRATSKWVAMNQDTRRLQKVNDKILEEIMSHAPRTPRLAFPEEDNCSLKKIPKLEEPAQYSKLGLKARRADLDMNQHVNNVTYIGWLLESLPQEIIDTHELRKVTLDYRRECQHDDTVDSLTSMELMDNAEAIPELNGTNGSATERKHEEDCCRFLHFVRFSGDGPEVNRGRTEWRRKSTR

SEQ ID NO: 25 - protein, 1050 aa, Aspergillus terreus
MIKETEQIPGPRPLPVVGNLFDMDLEHGLECLIRLADDFGPLFQITINGEKQIFATSQALVDELCDESRFHKAVMGGLEKLRMLASDGLFTAYHGERGWGIAHRILVPAFGPLRIRNMFEEMNDVAQQLCLKWARQGSSTSINITDDFTRLTLDTIALCTMNFRLNSFYNNETMHPFVKSMLYVLRESDIQSMLPGIANCIRVKARSRMSKHIQLMRNMARGIIQERRDQAEPVDDLLNTLLNGRDPVTGEGMSDDLIINNVITFLIAGHETTSGLLSFTFYYLLQNPHILERAQNEVDEVTGGERITVQHLGRLTYIDAILKESLRLMPTAPAFTVTPKKPEVLGGAWAIDAGQAVNVLLPVCLRDRSVFGPDADEFRPERMLEENFSKLPPNSWKPFGNGERSCIGRAFAWQEAQLVVAMVLQTFDLVPDDPSYKLRIKETLTIKPDGFRVRATLRRGQSATGLSQGSMSASGATSSVASPGPPAATGAQSNPAGGQRISFFYGSNSGTCKALAHRLASSLMGRGFTEQKLAALDTVVGNLPTDQPVIIVTTSYDGRPTDDAEEFVRWLESKRPVLQGVSYAVFGCGHHDWAKTFYRIPILIDDLMHKAGATRLTALGTANAAVSDLFSDLELWEETNLLPALREAFPPSNSSDVESSEPHQLQICVSKPRRVDMHRGLVEAKVTAVRTLTSPDSPEKRHVEFHVQGDTTWRPGDHVNILPVNPLSTVSRVLAYFQLAPDHSITVNSFNTQGLPSATPVSATELFSSFVELSQPATRKNLKALAMAAESKTDEQELIRLHDSYDALVRDKRVSVLDILERFPSISLPIGIFISMLPPLRLRTYSLSMAPSFKPSHGSLTFSVINEPAWSGNGQYLGVGSNYLASLTPGSLLYLSPRPAKDAFHLPADQFNTPIIMICAGSGLAPFMGFIQERMTWLKQGRPLAKGLLFFGCRGPHLDDLYYEELSEFEDAGVVEVHRAYSRAPDDVRAKGCRHVQHRLVTEAEAVRDHWGRNAIVYVCGSSNMARGVQTVLEEILGTLPPERYVAEIF

SEQ ID NO: 26 - protein, 1050 aa, Aspergillus kawachii
MKDAERIPGPKPLPVVGNLFDIDPEHSLESIVAFAEKFGPLFQITINGEKQIFATSQALVDELCDELRFHKAVVTGLEILRLLAHDGLFTAYHGERGWGIAHRILVPAFGPLRIRNMLDDMSDVAQQLCLKWARQGGSTSINITEDFTRLTLDTIALCTMGFRLNSFYNNETMHPFVQSMLYVLREADIQANLPGIANSIRVSAQRRMHKNIEAMRTMARGIIQERRKNKNPVDDILNTLLNGRDPVTGEGMSDDSIIDNVITFLIAGHETTSGLLSFTFYFLIQHPHILKKAQEEVDETVGLAQISAQHLAELPYIDAILKESLRLMPTAPGFTVTPKKTEVLGGRWMINAGQPVNVLLPACLRDQSVFGPDADEFRPERMLAENFSKLPPNSWKPFGNGERGCIGRAFAWQEAQLVVAMILQTFDLVPDDPSYQLRIKETLTIKPDGFRIRATLRRGQTATGLSRRSMLVARDGSSEESSNHPAEARGDHAPARGQPVSFFYGSNSGTCKALAHQLASNMMSRGYTTQKLAPLDNAVDNLPRDQPVIILTTTYDGQPTDNAKKFVAWLETGNVLSLQGISYAVFGCGHHDWTQTFYRIPILIDDLMYKAGATRLAPRGAANAAVSDLFSDLEAWEETSLLPALRENFLPSNSTDFDPLNPHQIQLSLSKPRRVDLHKGLIEAKVTAVRVLTSPDTPEKRHLEFCFQGDLSLRPGDHLNILPVNPPSTVSRVLAQFNLAPDYNITVNSFNTLGLPQATPVSASELFSSYVELCQPATRNNLKSLIAATQSDTVKQELNRLYDSYEFIVRDKRVSVLDLLEQFPSISLPIAAFISMLPALRVRTYSLSMAPAFKPSHSSLTFSVINEPAWRGSGQHLGVASNYLASLTSGSIFYFSPRPAKETFHLPKDPSRTPIIMICAGSGLAPFLSFIQDRMVLKQQNKPLAKAFLFFGCRGRSLDDLYHEELSEYEAAGVVEVRRAYSKTPEFDIAKGCRYVQHRLVTEGQAILSLWAQNAIIYVCGSTSMAKGAEAVLQNMLGPLPKERYVTEIF

SEQ ID NO: 27 - protein, 1091 aa, Aspergillus niger
MSMLLGVRGTRDQSSYIGGRDYVFWQKEMRDAERIPGPTPLPVVGNLFDIDLEHVLQSVIGLANKYGPLFQITINGEKQIFATSQALVDELCDESRFHKAVASGLENLRMLAHDGLFTAYHGERGWGIAHRILVPAFGPLRIQSMFDDMGDLAQQLCLKWARQGASNSINITDDFTRLTLDTIALCTMDFRLNSFYNNDTMHPFVESMLYVLREADVQSALPGIANSVRIMAHRRMLKNIEAMRTIARDIIHDRRKKENPADDLLNTLLNGRDPVTGEGMSDESIIDNVITFLVAGHETTSGLLSFTFYYLVQHPDILKKAQKEVDETVGQAQISVQHLAELPYIDAILKESLRMMPTAPGFTVTPKKAETLGGKWLLNAGQPINVLLPACLRDRSIFGPNADEFSPGRMLAENFSKLPPNSWKPFGNGERSCIGRAFAWQEAQLVVAMILQNFDLVPDDPSYTLRIKETLTIKPDGFRVRATLRHRQTATGLFQHTLSARNDTSLASSSAHFIKKSEDQAPAGGRPICFFYGSNSGTCKALAHRLASDLMPYGFTDQKLAVLDTAVDNLPRDQPVIILTTTYDGQPTDDAKKFVAWLESGKVPALQGISYAVFGCGHHDWTQTFYRIPTLIDELMHKAGATRLAPRGTANAAVSDLFSDLEAWEETSLLPALRETFLLSSSSDLEPLNLHQLQISLSKPRRIDLHKDLMEARVTTVRILTNPDTPEKRHIEFRFQGDTTLRPGDHVNVLPVNPPSTVLRVLAQFNLAPDYSITINSFNTLGLPQATPVSASELFSAYVELSQPATRNNLRILAATAQSDEDKQELIHLQDSYDSLVRDKRVSVLDLLEQFPSVSLPIAAFISMLPALRLRTYSLSLAPSFKPSHGSLTFSVVNEPARNGNRRYLGVGSNYLASLTPGSILYLSPRPAKEAFHLPVDQSRIPIIMICAGSGLAPFLSFIQDRMIWQQQDKPLARALLFFGCGGRFLDDLYHEELSEFEAAGVVDVRRAYSKVLDYDMARGCKYVQDRLVAEANAIRHLWAQDATIYVCGSADMAKGVEGVLEKLLGMLPRERYVTEIYQMQTRDNVSEWLI

SEQ ID NO: 28 - protein, 1048 aa, Penicillium expansum
MKDMESIPGPKPLPVVGNLFDIDLENGLQSIIKMAHEFGPLFQITINGQKQIFATSQALVDELCDETRFHKAVMGGIQKLRMLAKDGLFTAYHGERGWGIAHRILMPAFGPLRIRDMFEDMSDVAQQLCFKWARQGSSTSINICDDFTRLTLDTIALCTMGFRLNSYYNSNALHPFIESMLYVLKEAELQSTLPGVANCMRVKAQRRMSKHIDAMRSMARNLIEERRAKPEPVDDLLNTLLNGRDPITGEGMSDDLIISNIITFLIAGHETTSGLLSFTFYYLLQNQDVLERARNEVDEVTGVGPITVQHLAKLPYIDAIMKESLRLMPTAPAFTVTPQKPEVLGGKWMINTGDSVNLLLPVCLRDETVFGPDAGEFRPNRMLEENFSKLPPNSWKPFGNGERGCIGRAFAWQEAQLVVALVLRTFDLAAEDPYYKLRIKETLTIKPDGFRIRATLRHGKSATALSQHNISVGAAASPASSTYLAGNENGRDAAGGQPVSFFYGSNSGTCKALTHRLASTMMTRGFTDQNIAPLDSAVDNLPRDQPTIIITTTYDGQPTDDAKKFVAWLESGNSPSLQGVSYAVFGCGHQDWTKTFYRIPILIDNLMYKAGATRLATRGAANAAISDLFSDLEVWEETNLLPGLRESFYPPNNSNFVPLEPHQLQISINKPTRVGMHRDLIEAKVTAIRTLTSPGAPEKRHLEFCIPGETTLRPGDHLNILPVNPPSTVSRALARFNLAPDHSITFESSNALDLPQATPVSAAELFSSYLELSQPATRNNLKELASTTPSDGEKQELLHLYDSYDSLIRAKRASVLDLLEQFTSVTLPITTFISMLPALRVRTYSLSMAPSFKPLHYSLTFSVINEPAWNGNGRYLGVASNYLASLNLGSILYISPRPAKDAFHLPTDQSSKPIIMICAGSGLAPFRSFIQDRMLWQQQDKTLAKALLFFGCRSPQLDDLYHDELSQFEAAGVVEVRRAYSKVPNHYLAKGCRYVQHRLLTETETIQDMWAQDAIIYVCGSGNLAKGVKAVLESMLGTLYERYITEIF

SEQ ID NO: 29 - protein, 1050 aa, Aspergillus niger
MKDAERIPGPKPLPVVGNLLDIDPEHGLQSIIAFADKYGPLFQITINGEKQIFATSQALVDELCDESRFHKAVVTGLEVLRLLAHDGLFTAYHGERGWGIAHRILVPAFGPLRIRNMLDDMSDVAQQLCLKWARQGGSTSINITEDFTRLTLDTIALCTMGFRLNSFYNNETMHPFVQSMLYVLKEADVQANLPGIANSIRVSAQRRMHKNIEAMRTMARGIIQERRKNKNPVDDILNTLLNGRDPVTGEGMSDDSIIDNVITFLIAGHETTSGLLSFTFYFLIQHPHILKKAQEEVDETVGLAQISAQHLAELPYIDAILKESLRLMPTAPGFAVTPKKTEVLGGKWMINAGQPVNVLLPACLRDQSVFGPDADEFHPERMLAENFSKLPPNSWKPFGNGERGCIGRAFAWQEAQLVVAMILQTFDLVPDDPSYQLRIKETLTIKPDGFRIRALLRRGQTATGLSRRSMLVARDGSSGESSNHLAEARGDHAPARGQPVSFFYGSNSGTCKALAHQLASNMMSRGYTTQKLAPLDNAVGNLPRDQPVIILTTTYDGQPTDDAKKFVAWLETGNVPSLQGISYAVFGCGHHDWTQTFYRIPILIDDLMHKAGATRLAPRGAANAAVSDLFSDLEAWEETSLLPALRENFLPSNSTDFDPLNPHQIQLSLSKPRRVDLHKGLIEAKVTAVRVLTSPDTPEKRHLEFCFQGDTSLRPGDHLNILPVNPPSTVSRVLAQFNLAPDYNITVNSFNTLGLPQATPVSASELFSSYVELCQPATRNNLKALIAATQSDPDKQELNRLYDSYEFIVRDKRVSVLDLLEQFPSISLPIAAFISMLPALRVRTYSLSMAPSFKPSHSSLTFSVINEPAWRGSGQHLGVASNYLASLTSGSIFYFSPRPAKESFHLPKDPSNTPIIMICAGSGLAPFLSFIQDRMVLKQQYKPLAKAFLFFGCRGRSLDDLYHEELSEFEAAGVVEIRRAYSKTPDFDIAKGCRYVQHRLVTEGQAILSLWSQNATIYVCGSTNMAKGVEAVLQNMLGPLPKERYVTEIF

SEQ ID NO: 30 - protein, 1049 aa, Penicillium camemberti
MKDMDCIPGPKPLPVVGNLFDLDLDNALQSIIRMADEFGPLFQITINGQKQIFATSQALVDELCDETRFHKAVMGGVEKLRMLAQDGLFTAHHGERGWGIAHRILMPAFGPLRIRDMFEDMSDVAHQLCFKWARQGSSASINIAEDFTRLTLDTIALCTMSFRLNSYYNSETMHPFVQSMLYVLKEADLQATLPGVANCVRVKAQRRMSKHIQAMRNIAGDIIKGRRDKPEPVDDLLNTLLNGRDPVTGEGMSDELIISNIITFLVAGHETTSGLLSFTFYYLLQHPHVLEQARNEVDEVVGVGPITVQHLAKLPYIDAVMKESLRLMPTAPAFTVTPKKPEVVGGKWMVNTGQSVHVLLPVCLRDEAVFGPDAGEFRPTRMLEENFSKLPPNSWKPFGNGERGCIGRAFAWQEAQLVVASVLQTFDLVAEDPYYKLRIKETLTIKPDGFRVRATLRRGQSATALSQHNMSAGATASPGSSTHLAGDENGQDTAGGQPISFFYGSNSGTCKALAHRLASTMMTRGFTDQHLAQLDSAVDNLPRDQPTIIVTTTYDGQPTDDAKKFLAWLESGNVPSLHGVSYAVFGCGHQDWTKTFYRIPILIDDLMHKAGATRLTTRGTANAAVSDLFSDLEVWEETNLLPALREKFYLCNSSDFEPLDPHQLQISISKPARVGMHRDLVEGKVTAIRTLTSPGVPEKRHVEFQIPSEMALRPGDHVNILPVNPPCSVLRALARFSLASDHSITFESSNALDLPQATPVSAAELFSSYLELSQPATRINLKSLASATPSDDDKKELLHFHDSYDSLIRDKRVSVLDLLEHFTSITLPIATFISMLPVLRVRTYSLSMAPSFKPLHCSLTFSVVNEPAWSGNGRYLGVGSNYLASLTPGSILYVSPRPAKDAFHLPTDQSSNPIIMICAGSGLAPFRSFIQDRMAWLQQGKPLAKALLFFGCRGPHLDDLYHDELSEFESAGVVEVRRAYSKVPNHYLAKGCRYAQHRLLTETETIQDMWAHNATLYLCGSANLAKGVKAVLENMLGTLSEERYITEIF

SEQ ID NO: 31 - protein, 1049 aa, Penicillium freii
MKDMDCIPGPKPLPVVGNLFDLDLDNALQSIIKMADEFGPLFQITVNRQKHIFATSQALVDELCDETRFHKAVIGGVEKLRMLAHDGLFTAHHGERGWGIAHRILMPAFGPLRIRDMFEDMSDVAHQLCFKWARQGSSTSINISEDFTRLTLDTIALCTMSFRLNSYYNSDTMHPFVQSMLYVLKEADLQSSLPEVANCVRVKAQRSMSKHIEAMRSIAGDIIKGRRDKPEPVNDLLNTLLNGRDPVTGEGMSDELIISNIITFLIAGHETTSGLLSFTFYYLLQHPQVLEQARNEVDEVVGVGPITVQHLAKLPYIDAIMKESLRLMPTAPSFTVTPKKPEVLGGKWMINPGQSVHVLLPVCLRDEAVFGPDAGEFRPNRMLEENFSKLPPNSWKPFGNGERGCIGRAFAWQEAQLVVASVLQTFDLVAEDPNYKLRVKETLTIKPDGFRVRATLRHGRSATALSQHNMSAGATSSPGSSAHPAGNKNAQDAAGGQSISFFYGSNSGTCKALAHRLASTMMTRGFTDQHLAPLDSAVDNLPKDQPTIIVTTTYEGQPTDDAKKFLAWLESGIVPSLHGVSYAVFGCGHQDWTKTFYRIPILIDDLMHKAGATRLTTRGEANAAVSDLFSDLEVWEETNLLPALREKFDASNSGEFESLDLQQLQISISKPTRVGMHRDLIEGKVTAIRTLTSPGVPEKRHVEFQITSDTTLRPGDHVNILPVNPPSTVLRALARFNLASDHIITFESSNALDLPQATPVSAAELFGSYLELSQPATRNNLKSLASTTPSDEDKQELLRFHDSYDSLIRDKRVSVLDLLEHFTSITLPIATFISMLPVLRVRTYSLSMAPSFKPLHCSLTFSVVNEPAWSGNGRYLGVGSNYLASLTPGSILYVSPRPAKEAFHLPADQSSKPIIMICAGSGLAPFRSFIQDRMAWLQQGKPLAKALLFFGCRGPQLDDLYHDELSEFESAGVVEVRRAYSKVPNHYPGKGCRYVQHRLFAETETIQDMWAHNATLYLCGSATLAKGVKATLENMLGTLSEERYITEIF

SEQ ID NO: 32 - protein, 127 aa, synthetic construct
  REGION 1..127: note = Description of Artificial Sequence: Synthetic polypeptide
  VARIANT 2: note = Asn, Ser or absent
  VARIANT 4: note = Thr, Lys, Met or Asn
  VARIANT 27: note = Ser or Arg
  VARIANT 65: note = Glu or Asp
  VARIANT 75: note = Met or Leu
  VARIANT 88: note = Thr or Ala
  VARIANT 91: note = Val or Thr
  VARIANT 101: note = Asn or Gly
  VARIANT 103: note = Leu or Val
  VARIANT 112: note = Val or Ala
MXTXLFRWPVRVYYEDTDAGGVVYHAXYVAFYERARTEMLRHHHFSQQVLLAERVAFVVRKMTVXYYAPARLDDXLEVQTEITSMRGXSLXFTQRIVNAEXTXLNEAEVLIXCVDPIKMKPRALPKS

(19) United States
(12) Patent Application Publication
(10) Pub. No.: US 2026/0193684 A1
(43) Pub. Date: Jul. 9, 2026

(54) Enzymatic Biosynthesis Of Lactones

(71) Applicant: Genomatica, Inc., San Diego, CA (US)

(72) Inventors: Andreas W. SCHIRMER, San Diego, CA (US); Katherine Ann MURPHY, San Diego, CA (US); Angelica Zabala BAUTISTA, San Diego, CA (US)

(21) Appl. No.: 19/562,232

(22) Filed: Mar. 10, 2026

Related U.S. Application Data
(63) Continuation of application No. 17/420,908, filed on Jan. 15, 2020, now Pat. No. 12,595,496.
(60) Provisional application No. 62/794,497, filed on Jan. 18, 2019.

(51) Int. Cl. C12P 17/06; C12N 9/00; C12N 9/16

(52) U.S. Cl. CPC C12P 17/06; C12N 9/16; C12N 9/93; C12Y 301/0202; C12Y 602/01003

(57) ABSTRACT

The disclosure relates to methods for the production of natural lactones by bacteria under physiological conditions. The methods employ ybgC proteins having lactonizing activity.

The present application claims the benefit of and priority to U.S. Provisional Application 62/794,497, filed on Jan. 18, 2019, the contents of which is incorporated herein by reference in its entirety

### FIELD

The disclosure relates to the field of specialty chemicals and methods for their synthesis. The disclosure provides bacteria engineered to produce lactones in vivo at neutral pH. Accordingly, the disclosure provides biochemical pathways, recombinant microorganisms and methods for the biological production of various lactones.

### BACKGROUND

Lactones are cyclic esters and are components of many flavors and fragrances. Flavors and fragrances are integral components of a wide range of consumer goods.

There is an increasing demand for natural flavor and fragrance ingredients to satisfy increasing demand in e.g., the food, beverage, fragrance and cosmetics industries.

Increasing demand for natural flavors and fragrances, requires that new methods be developed to meet this increasing demand. To be natural, compounds have to result from the extraction of natural materials and/or to be transformed by natural means e.g., through the use of enzymes or whole cells (see e.g., EFFA Guidance Document for the Production of Natural Flavouring Substances and (Natural) Flavouring Preparations in the EU).

Thus, natural lactones containing 8 to 14 carbon atoms, such as e.g. γ-decalactone or δ-dodecalactone, can be extracted from their natural sources, e.g. from fruits like peach, strawberry or pineapple. Natural lactones can also be prepared from microbial cells e.g., from yeast see e.g., However, biotechnological processes that involve a chemical conversion step e.g. the bioconversion of ricinoleic acid or other oleochemicals using certain yeast strains (see e.g., Sushilkumar et al., 2008 Advanced Biotech, p. 20-30; Kourist and Hilterhaus 2015, p. 275-301, in Microorganisms in Biorefineries, B. Kamm (ed.), Springer Verlag), disqualifies the lactone product from being marketed and sold as natural flavor (see e.g., EFFA Guidance Document supra).

Therefore, what is needed in the art are processes for the production of natural lactones e.g., γ-, δ- and/or ε-lactones that meet strict requirements for manufacturing of natural flavors.

Fortunately, as will be clear from the detailed description that follows, the present disclosure provides for this and other needs.

### SUMMARY

In one aspect the disclosure provides a method for enzymatically producing a lactone under physiological conditions, the method comprising: culturing a recombinant bacterium that heterologously expresses a YbgC protein and an acyl-CoA synthetase protein, in a culture medium having a neutral pH, wherein the heterologously expressed YbgC protein converts an hydroxy acyl-CoA substrate to a lactone, and wherein the hydroxyl acyl-CoA substrate is a member selected from: a 4-hydroxy acyl-CoA; a 5-hydroxy acyl-CoA and a 6-hydroxy acyl-CoA, and wherein the heterologously expressed YbgC protein converts the 4-hydroxy acyl-CoA to a γ-lactone; the heterologously expressed YbgC protein converts the 5-hydroxy acyl-CoA to a 8-lactone and the heterologously expressed YbgC protein converts the 6-hydroxy acyl-CoA, to an ε-lactone.

In embodiments, the heterologously expressed YbgC protein is overexpressed.

In embodiments, the hydroxy acyl-CoA substrate is produced by the recombinant bacterium from a fatty acid derivative molecule exogenously added to the culture medium. In embodiments, the fatty acid derivative molecule exogenously added to the culture medium is a 4-hydroxy fatty acid derivative, a 5-hydroxyl fatty acid derivative or a 6-hydroxy fatty acid derivative. In embodiments, the fatty acid derivative molecule exogenously added to the culture medium is a 4-hydroxy fatty acid derivative and the recombinant bacterium produces the 4-hydroxy acyl-CoA from the 4-hydroxy fatty acid derivative. In embodiments, the 4-hydroxy fatty acid derivative is 4-hydroxy decanoic acid.

In embodiments, the recombinant bacterium produces the 5-hydroxy acyl-CoA from the 5-hydroxyl fatty acid derivative exogenously added to the culture medium. In embodiments, the 5-hydroxy fatty acid derivative exogenously added to the culture medium is 5-hydroxy decanoic acid.

In embodiments, the recombinant bacterium produces 6-hydroxy acyl-CoA from the 6-hydroxy fatty acid derivative exogenously added to the culture medium. In embodiments, the 6-hydroxy fatty acid derivative is 6-hydroxy hexanoic acid.

In embodiments, the heterologously expressed YbgC protein used in the method for enzymatically producing a lactone under physiological conditions wherein the fatty acid derivative molecule is exogenously added to the culture medium has at least 60% sequence identity to SEQ ID NO: 1 over the full length of the YbgC amino acid sequence.

In embodiments, the heterologously expressed YbgC protein used in the method for enzymatically producing a lactone under physiological conditions wherein the fatty acid derivative molecule is exogenously added to the culture medium has at least 70% sequence identity to SEQ ID NO:1 over the full length of the YbgC amino acid sequence.

In embodiments, the heterologously expressed YbgC protein used in the method for enzymatically producing a lactone under physiological conditions wherein the fatty acid derivative molecule is exogenously added to the culture medium has at least 75% sequence identity to SEQ ID NO: 1 over the full length of the YbgC amino acid sequence.

In embodiments, the heterologously expressed YbgC protein used in the method for enzymatically producing a lactone under physiological conditions wherein the fatty acid derivative molecule is exogenously added to the culture medium has at least 80% sequence identity to SEQ ID NO: 1 over the full length of the YbgC amino acid sequence.

In embodiments, the heterologously expressed YbgC protein used in the method for enzymatically producing a lactone under physiological conditions wherein a fatty acid derivative molecule is exogenously added to the culture medium has at least 85% sequence identity to SEQ ID NO: 1 over the full length of the YbgC amino acid sequence.

In embodiments, the heterologously expressed YbgC protein used in the method for enzymatically producing a lactone under physiological conditions wherein the fatty acid derivative molecule is exogenously added to the culture medium has at least 90% sequence identity to SEQ ID NO: 1 over the full length of the YbgC amino acid sequence.

In embodiments, the heterologously expressed YbgC protein used in the method for enzymatically producing a lactone under physiological conditions wherein the fatty acid derivative molecule is exogenously added to the culture medium has at least 95% sequence identity to SEQ ID NO: 1 over the full length of the YbgC amino acid sequence.

In embodiments, the heterologously expressed YbgC protein used in the method for enzymatically producing a lactone under physiological conditions wherein the fatty acid derivative molecule is exogenously added to the culture medium is SEQ ID NO:1.

In embodiments, the heterologously expressed YbgC protein that has at least 60% sequence identity to SEQ ID NO: 1 over the full length of the YbgC amino acid sequence that is used in the method for enzymatically producing a lactone under physiological conditions wherein the fatty acid derivative molecule is exogenously added to the culture medium is selected from the group consisting of: Escherichia coli ybgC (SEQ ID NO: 1); Citrobacter koseri ybgC (SEQ ID NO:2); Enterobacter cloacae ybgC (SEQ ID NO: 3); Serratia fonticola ybgC (SEQ ID NO:4); Exiguobacterium mexicanum ybgC (SEQ ID NO: 5) and Plesiomonas shigelloides ybgC (SEQ ID NO:6).

In one aspect the disclosure provides a method for enzymatically producing a lactone under physiological conditions, the method comprising: culturing a recombinant bacterium that heterologously expresses a YbgC protein, an acyl-CoA synthetase protein, a thioesterase and an hydroxylating enzyme, in a culture medium having a neutral pH, wherein the heterologously expressed YbgC protein converts an hydroxy acyl-CoA substrate to a lactone, and wherein the hydroxyl acyl-CoA substrate is a member selected from: a 4-hydroxy acyl-CoA; a 5-hydroxy acyl-CoA and a 6-hydroxy acyl-CoA, and wherein the heterologously expressed YbgC protein converts the 4-hydroxy acyl-CoA to a γ-lactone; the heterologously expressed YbgC protein converts the 5-hydroxy acyl-CoA to a δ-lactone and the heterologously expressed YbgC protein converts the 6-hydroxy acyl-CoA, to an ε-lactone.

In embodiments, the thioesterase is a member selected from SEQ ID NO: 16, SEQ ID NO:17, SEQ ID NO: 18, SEQ ID NO: 19, SEQ ID NO:20, SEQ ID NO:21, SEQ ID NO: 22, SEQ ID NO:23 and SEQ ID NO:24, and the hydroxylating enzyme is a member selected from SEQ ID NO:25, SEQ ID NO:26, SEQ ID NO:27, SEQ ID NO:28, SEQ ID NO: 29, SEQ ID NO:30 and SEQ ID NO:31.

In embodiments, the recombinant bacterium produces the lactone from a simple carbon source.

In embodiments, the heterologously expressed YbgC protein used in the method for enzymatically producing a lactone under physiological conditions from a simple carbon source, wherein the method comprises culturing a recombinant bacterium that heterologously expresses a YbgC protein, an acyl-CoA synthetase protein, a thioesterase and an hydroxylating enzyme, in a culture medium having a neutral pH, has at least 60% sequence identity to SEQ ID NO: 1 over the full length of the YbgC amino acid sequence.

In embodiments, the heterologously expressed YbgC protein used in the method for enzymatically producing a lactone under physiological conditions from a simple carbon source, wherein the method comprises culturing a recombinant bacterium that heterologously expresses a YbgC protein, an acyl-CoA synthetase protein, a thioesterase and an hydroxylating enzyme, in a culture medium having a neutral pH, has at least 70% sequence identity to SEQ ID NO:1 over the full length of the YbgC amino acid sequence.

In embodiments, the heterologously expressed YbgC protein used in the method for enzymatically producing a lactone under physiological conditions from a simple carbon source, wherein the method comprises culturing a recombinant bacterium that heterologously expresses a YbgC protein, an acyl-CoA synthetase protein, a thioesterase and an hydroxylating enzyme, in a culture medium having a neutral pH, has at least 75% sequence identity to SEQ ID NO: 1 over the full length of the YbgC amino acid sequence.

In embodiments, the heterologously expressed YbgC protein used in the method for enzymatically producing a lactone under physiological conditions from a simple carbon source, wherein the method comprises culturing a recombinant bacterium that heterologously expresses a YbgC protein, an acyl-CoA synthetase protein, a thioesterase in and an hydroxylating enzyme, in a culture medium having a neutral pH, has at least 80% sequence identity to SEQ ID NO:1 over the full length of the YbgC amino acid sequence.

In embodiments, the heterologously expressed YbgC protein used in the method for enzymatically producing a lactone under physiological conditions from a simple carbon source, wherein the method comprises culturing a recombinant bacterium that heterologously expresses a YbgC protein, an acyl-CoA synthetase protein, a thioesterase and an hydroxylating enzyme, in a culture medium having a neutral pH, has at least 85% sequence identity to SEQ ID NO: 1 over the full length of the YbgC amino acid sequence.

In embodiments, the heterologously expressed YbgC protein used in the method for enzymatically producing a lactone under physiological conditions from a simple carbon source, wherein the method comprises culturing a recombinant bacterium that heterologously expresses a YbgC protein, an acyl-CoA synthetase protein, a thioesterase and an hydroxylating enzyme, in a culture medium having a neutral pH, has at least 90% sequence identity to SEQ ID NO: 1 over the full length of the YbgC amino acid sequence.

In embodiments, the heterologously expressed YbgC protein used in the method for enzymatically producing a lactone under physiological conditions from a simple carbon source, wherein the method comprises culturing a recombinant bacterium that heterologously expresses a YbgC protein, an acyl-CoA synthetase protein, a thioesterase and an hydroxylating enzyme, in a culture medium having a neutral pH, has at least 95% sequence identity to SEQ ID NO: 1 over the full length of the YbgC amino acid sequence.

In embodiments, the heterologously expressed YbgC protein used in the method for enzymatically producing a lactone under physiological conditions from a simple carbon source, wherein the method comprises culturing a recombinant bacterium that heterologously expresses a YbgC protein, an acyl-CoA synthetase protein, a thioesterase and an hydroxylating enzyme, in a culture medium having a neutral pH, is SEQ ID NO:1.

In embodiments, the heterologously expressed YbgC protein used in the method for enzymatically producing a lactone under physiological conditions from a simple carbon source, wherein the method comprises culturing a recombinant bacterium that heterologously expresses a YbgC protein, an acyl-CoA synthetase protein, a thioesterase and an hydroxylating enzyme, in a culture medium having a neutral pH, that has at least 60% sequence identity to SEQ ID NO: 1 over the full length of the YbgC amino acid sequence is a member selected from the group consisting of: Escherichia coli ybgC (SEQ ID NO: 1); Citrobacter koseri ybgC (SEQ ID NO:2); Enterobacter cloacae ybgC (SEQ ID NO: 3); Serratia fonticola ybgC (SEQ ID NO:4); Exiguobacterium mexicanum ybgC (SEQ ID NO: 5) and Plesiomonas shigelloides ybgC (SEQ ID NO:6).

Other features, objects and advantages of the invention will be apparent from the detailed description which follows.

### BRIEF DESCRIPTION OF THE FIGURES

FIG. 1 Illustrates a biochemical pathway converting an oleochemical to γ-decalactone. When the last step is carried out enzymatically, the product is natural γ-decalactone.

FIG. 2 Illustrates a biochemical pathway converting 4-hydroxydecanoic acid to natural γ-decalactone (A) or 5-hydroxydecanoic acid to natural δ-decalactone (B), or 6-hydroxyhexanoic acid to natural ¿-lactone.

FIG. 3 Illustrates a biochemical pathway converting a simple carbon source to natural γ-decalactone via a medium chain thioester (decanoyl-ACP).

FIG. 4 Illustrates a Illustrates a biochemical pathway converting a simple carbon source to natural γ-decalactone via a long-chain acyl-thioester (palmitoleyl-ACP).

FIG. 5 Illustrates a GC/MS chromatograph of extracts from recombinant E. coli strains (B-D) when fed with 4-hydroxy decanoic acid, which was efficiently converted to γ-decalactone by strain sKM.529 expressing FadD3 from P. putida and YbgC from E. coli. Authentic external and internal standards are shown in (A).

FIG. 6 Illustrates the mass spectrum and ion fragmentation pattern of γ-decalactone authentic standard and the peak at RT=6.9 min from FIG. 3D, minutes), which are identical.

FIG. 7 Illustrates a GC/MS chromatograph of extracts from recombinant E. coli strains (B-D) when fed with 5-hydroxy decanoic acid, which was efficiently converted to γ-decalactone by strain sKM.529 expressing FadD3 from P. putida and YbgC from E. coli. Authentic external and internal standards are shown in (A).

FIG. 8 Illustrates the mass spectrum and ion fragmentation pattern of 8-decalactone authentic standard and the peak at RT=6.9 min from FIG. 3D, minutes), which are identical.

FIG. 9 Illustrates an alignment of six ybgC proteins (SEQ ID NO; 1-SEQ ID NO: 6) which have lactonizing activity.

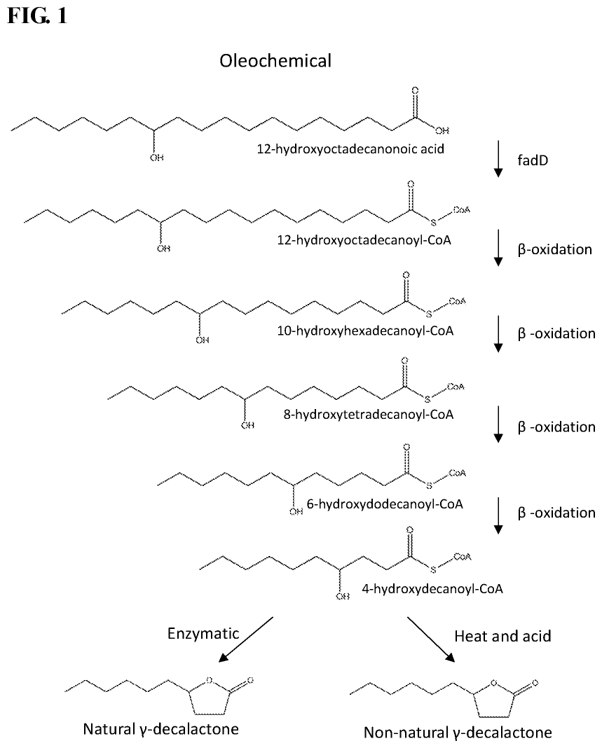

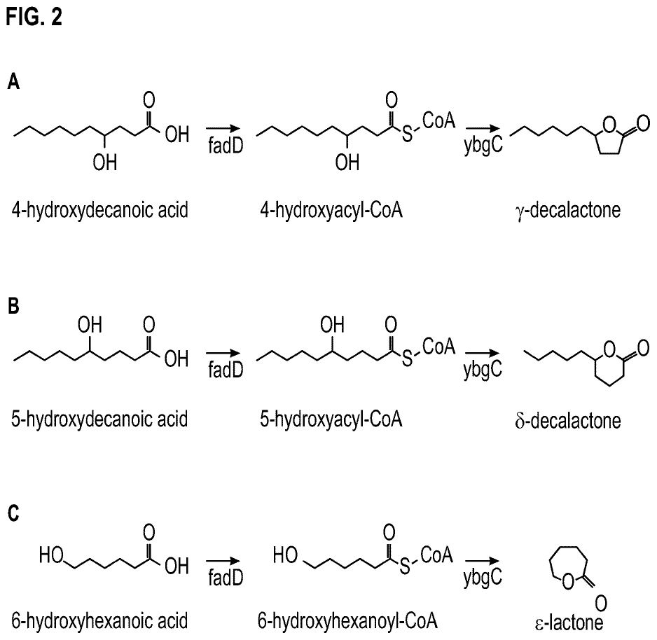

### DETAILED DESCRIPTION

### Definitions

As used herein and in the appended claims, singular articles such as “a” and “an” and “the” and similar referents in the context of describing the elements are to be construed to cover both the singular and the plural, unless otherwise indicated herein or clearly contradicted by context.

As used herein, “about” is understood by persons of ordinary skill in the art and may vary to some extent depending upon the context in which it is used. If there are uses of the term which are not clear to persons of ordinary skill in the art given the context in which the term “about” is used, “about” will mean up to plus or minus 10% of the particular term.

As will be understood by one skilled in the art, for any and all purposes, all ranges disclosed herein also encompass any and all possible subranges and combinations of subranges thereof. Furthermore, as will be understood by one skilled in the art, a range includes each individual member. Thus, for example, a group having 1-3 atoms refers to groups having 1, 2, or 3 atoms. Similarly, a group having 1-5 atoms refers to groups having 1, 2, 3, 4, or 5 atoms, and so forth.

Unless defined otherwise, technical and scientific terms used herein have the same meaning as commonly understood by a person of ordinary skill in the art. In particular, this disclosure utilizes routine techniques in the field of recombinant genetics, organic chemistry, fermentation and biochemistry. Basic texts disclosing the general terms in molecular biology and genetics include e.g., Lackie, Dictionary of Cell and Molecular Biology, Elsevier (5th ed. 2013). Basic texts disclosing methods in recombinant genetics and molecular biology include e.g., Sambrook et al., Molecular Cloning—A Laboratory Manual, Cold Spring Harbor Press 4^{th} Edition (Cold Spring Harbor, N.Y. 2012) and Current Protocols in Molecular Biology Volumes 1-3, John Wiley & Sons, Inc. (1994-1998) and Supplements 1-115 (1987-2016). Basic texts disclosing the general methods and terms in biochemistry include e.g., Lehninger Principles of Biochemistry sixth edition, David L. Nelson and Michael M. Cox eds. W. H. Freeman (2012). Basic texts disclosing the general methods and terminology of fermentation include e.g., Principles of Fermentation Technology, 3rd Edition by Peter F Stanbury, Allan Whitaker and Stephen J Hall. Butterworth-Heinemann (2016). Basic texts disclosing the general methods and terms organic chemistry include e.g., Favre, Henri A. and Powell, Warren H. Nomenclature of Organic Chemistry. IUPAC Recommendations and Preferred Name 2013. Cambridge, UK: The Royal Society of Chemistry, 2013; Practical Synthetic Organic Chemistry: Reactions, Principles, and Techniques, Stephane Caron ed., John Wiley and Sons Inc. (2011); Organic Chemistry, 9th Edition—Francis Carey and Robert Giuliano, McGraw Hill (2013).

Those of skill in the art will appreciate that compounds of the present technology may exhibit the phenomena of tautomerism, conformational isomerism, geometric isomerism and/or stereoisomerism. As the formula drawings within the specification and claims can represent only one of the possible tautomeric, conformational isomeric, stereochemical or geometric isomeric forms, it should be understood that the present technology encompasses any tautomeric, conformational isomeric, stereochemical and/or geometric isomeric forms of the compounds having one or more of the utilities described herein, as well as a mixture of these various different forms.

“Tautomers” refers to isomeric forms of a compound that are in equilibrium with each other. The presence and concentrations of the isomeric forms will depend on the environment the compound is found in and may be different depending upon, for example, whether the compound is a solid or is in an organic or aqueous solution. For example, in aqueous solution, quinazolinones may exhibit the following isomeric forms, which are referred to as tautomers of each other:

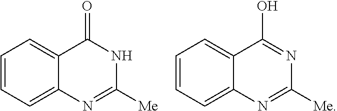

As another example, guanidines may exhibit the following isomeric forms in protic organic solution, also referred to as tautomers of each other:

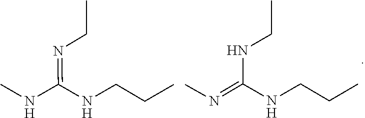

Because of the limits of representing compounds by structural formulas, it is to be understood that all chemical formulas of the compounds described herein represent all tautomeric forms of compounds and are within the scope of the present technology.

Stereoisomers of compounds (also known as optical isomers) include all chiral, diastereomeric, and racemic forms of a structure, unless the specific stereochemistry is expressly indicated. Thus, compounds used in the present technology include enriched or resolved optical isomers at any or all asymmetric atoms as are apparent from the depictions. Both racemic and diastereomeric mixtures, as well as the individual optical isomers can be isolated or synthesized so as to be substantially free of their enantiomeric or diastereomeric partners, and these stereoisomers are all within the scope of the present technology.

Geometric isomers can be represented by the symbol which denotes a bond that can be a single, double or triple bond as described herein. Provided herein are various geometric isomers and mixtures thereof resulting from the arrangement of substituents around a carbon-carbon double bond. Substituents around a carbon-carbon double bond are designated as being in the “Z” or “E” configuration wherein the terms “Z” and “E” are used in accordance with IUPAC standards. Unless otherwise specified, structures depicting double bonds encompass both the “E” and “Z” isomers.

Substituents around a carbon-carbon double bond alternatively can be referred to as “cis” or “trans,” where “cis” represents substituents on the same side of the double bond and “trans” represents substituents on opposite sides of the double bond. The term “cis” represents substituents on the same side of the plane of the ring, and the term “trans” represents substituents on opposite sides of the plane of the ring. Mixtures of compounds wherein the substituents are disposed on both the same and opposite sides of plane of the ring are designated “cis/trans.”

In certain embodiments, the pharmaceutically acceptable form thereof is an isomer. “Isomers” are different compounds that have the same molecular formula. “Stereoisomers” are isomers that differ only in the way the atoms are arranged in space. As used herein, the term “isomer” includes any and all geometric isomers and stereoisomers. For example, “isomers” include cis- and trans-isomers, E- and Z-isomers, R- and S-enantiomers, diastereomers, (d)-isomers, (1)-isomers, racemic mixtures thereof, and other mixtures thereof, as falling within the scope of this disclosure.

“Enantiomers” are a pair of stereoisomers that are non-superimposable mirror images of each other. A mixture of a pair of enantiomers in any proportion can be known as a “racemic” mixture. The term “(+−)” is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer, the stereochemistry at each chiral carbon can be specified by either R or S. Resolved compounds whose absolute configuration is unknown can be designated (+) or (−) depending on the direction (dextro- or levorotatory) which they rotate plane polarized light at the wavelength of the sodium D line. Certain of the compounds described herein contain one or more asymmetric centers and can thus give rise to enantiomers, diastereomers, and other stereoisomeric forms that can be defined, in terms of absolute stereochemistry, as (R)- or (S)-. The present chemical entities, pharmaceutical compositions and methods are meant to include all such possible isomers, including racemic mixtures, optically pure forms and intermediate mixtures. Optically active (R)- and (S)-isomers can be prepared, for example, using chiral synthons or chiral reagents, or resolved using conventional techniques. The optical activity of a compound can be analyzed via any suitable method, including but not limited to chiral chromatography and polarimetry, and the degree of predominance of one stereoisomer over the other isomer can be determined.

The term “fatty acid” as used herein, refers to an aliphatic carboxylic acid having the formula RCOOH wherein R is an aliphatic group having at least 4 carbons, typically between about 4 and about 28 carbon atoms. The aliphatic R group can be saturated or unsaturated, branched or unbranched. Unsaturated “fatty acids” may be monounsaturated or polyunsaturated.

A “fatty acid” or “fatty acids”, as used herein, can be produced within a cell through the process of fatty acid biosynthesis, through the reverse of fatty acid degradation or beta-oxidation, or they can be fed to a cell. As is well known in the art, fatty acid biosynthesis is generally a malonyl-CoA dependent synthesis of acyl-ACPs or acyl CoAs, while the reverse of beta-oxidation results is acetyl-CoA dependent and results in the synthesis of acyl-CoAs. Fatty acids fed to cell are converted to acyl-CoAs and can be converted to acyl-ACPs. Fatty acids can be synthesized in a cell by natural fatty acid biosynthetic pathways or can be synthesized from heterologous fatty acid biosynthetic pathways that comprise a combination of fatty acid biosynthetic and/or degradation enzymes that result in the synthesis of acyl-CoAs and/or Acyl-ACPs.

Fatty acid biosynthesis and degradation occur in all life forms, including prokaryotes, single cell eukaryotes, higher eukaryotes, and Archaea. The tools and methods disclosed herein are useful in the production of fatty acid derivatives that are derived through any one or more of fatty acid synthesis, degradation, or feeding in any organism that naturally produces alkyl thioesters.

The term “fatty acid derivative” as used herein, refers to a product derived from a fatty acid. Thus, a “fatty acid derivative” includes “fatty acids” as defined above. In general, “fatty acid derivatives” include malonyl-CoA derived compounds including acyl-ACP or acyl-ACP derivatives. “Fatty acid derivatives” also include malonyl-CoA derived compounds such as acyl-CoA or acyl-CoA derivatives. “Fatty acid derivatives” also include acetyl-CoA derived compounds such as acyl-CoA or acyl-CoA derivatives. Thus, a “fatty acid derivatives” include alkyl-thioesters and acyl-thioesters. Further, a “fatty acid derivative” includes a molecule/compound that is derived from a metabolic pathway that includes a fatty acid derivative enzyme. Exemplary fatty acid derivatives include e.g., natural lactones such as e.g., γ-, δ-, and/or ε-lactones as disclosed herein, fatty acids, fatty acid esters (e.g., waxes, fatty acid esters, fatty acid methyl esters (FAME), fatty acid ethyl esters (FAEE)), fatty alcohol acetate esters (FACE), fatty amines, fatty aldehydes, fatty alcohols, hydrocarbons e.g., alkanes, alkenes, etc, ketones, terminal olefins, internal olefins, 3-hydroxy fatty acid derivatives, bifunctional fatty acid derivatives (e.g., ω-hydroxy fatty acids, (ω-1)-hydroxy fatty acids, (ω-2)-hydroxy fatty acids, (ω-3)-hydroxy fatty acids, 10-hydroxy fatty acids, 1,3 fatty-diols, α,ω-diols, α,α-3-hydroxy triols,-hydroxy FAME, ω-OH FAEE, etc.), unsaturated fatty acid derivatives, including unsaturated compounds of each of the above mentioned fatty acid derivatives, etc.

The expression “natural lactone” as used herein, refers to a lactone produced in its entirety by an organism under physiological conditions. A natural lactone is produced in its final form by an organism, without any need for chemical processing (such as e.g, acidic conditions and/or increased temperatures) to produce the lactone. Thus, in embodiments, “natural lactones” are lactones produced by recombinant bacteria that heterologously express a ybgC protein. Such “natural lactones” are isolated from the recombinant bacteria in their final form and require no chemical processing to make. Thus, “natural lactones” are “enzymatically produced lactones” since such “natural lactones” are produced enzymatically by an organism without any need for chemical processing.

The expression “fatty acid derivative composition” as used herein, refers to a composition of fatty acid derivatives, as disclosed herein. A “fatty acid derivative composition” may comprise a single fatty acid derivative species or may comprise a mixture of fatty acid derivative species. In some embodiments, the mixture of fatty acid derivatives includes more than one type of fatty acid derivative product (e.g., natural lactones, fatty acids, fatty acid esters, fatty alcohols, fatty alcohol acetates, fatty aldehydes, fatty amine, bifunctional fatty acid derivatives, and non-native monounsaturated fatty acid derivatives, etc.). In other exemplary embodiments, the mixture of fatty acid derivatives includes fatty acid derivatives with different chain lengths, saturation and/or branching characteristics. In other exemplary embodiments, the mixture of fatty acid derivatives comprises predominantly one type of fatty acid derivative e.g., a composition of natural lactones e.g., γ-lactones, δ-lactones and/or ε-lactones. In still other exemplary embodiments, a “fatty acid derivative composition” comprises a mixture of more than one type of fatty acid derivative product e.g., fatty acid derivatives with different chain lengths, saturation and/or branching characteristics. In still other exemplary embodiments, a “fatty acid derivative composition” comprises a mixture of fatty esters and 3-hydroxy esters. In still other exemplary embodiments, a fatty acid derivative composition comprises a mixture of fatty alcohols and fatty aldehydes. In still other exemplary embodiments, a fatty acid derivative composition comprises a mixture of FAME and/or FAEE. In still other exemplary embodiments, a fatty acid derivative composition comprises a mixture of fatty alcohol acetate esters (FACE), for example a mixture of non-native monounsaturated fatty alcohol acetate esters (FACE).

The term “malonyl-CoA derived compound” as used herein refers to any compound or chemical entity (i.e., intermediate or end product) that is made via a biochemical pathway wherein malonyl-CoA functions as intermediate and/or is made upstream of the compound or chemical entity. For example, a malonyl-CoA derived compound may include, but is not limited to, a fatty acid derivative such as, for example, natural lactone e.g., a natural γ-, δ-, and/or ε-lactone, a fatty acid; a fatty ester including, but not limited to a fatty acid methyl ester (FAME) and/or a fatty acid ethyl ester (FAEE); a fatty alcohol; a fatty aldehyde; a fatty amine; an alkane; an olefin or alkene; a hydrocarbon; a beta hydroxy fatty acid derivative, a bifunctional fatty acid derivative, a multifunctional fatty acid derivative, a native or non-native unsaturated fatty acid derivative, etc.

As used herein “alkyl-thioester” or equivalently an “acyl thioester” is a compound in which the carbonyl carbon of an acyl chain and the sulfydryl group of an organic thiol are joined through a thioester bond. Representative organic thiols include e.g., Cystein, beta-cysteine, glutathione, mycothiol, pantetheine, Coenzyme A (CoA) and the acyl carrier protein (ACP). An “acyl-ACP” refers to an “alkyl-thioester” formed between the carbonyl carbon of an acyl chain and the sulfhydryl group of the phosphopantetheinyl moiety of an ACP. An “Acyl-CoA” refers to an “alkyl-thioester” formed between the carbonyl carbon of an acyl chain and the sulfhydryl group of the phosphopantetheinyl moiety of CoA. In some embodiments an “alkyl-thioester”, such as acyl-ACP or acyl CoA, is an intermediate in the synthesis of fully saturated acyl-thioesters. In other embodiments an “alkyl-thioester”, such as acyl-ACP or acyl-CoA, is an intermediate in the synthesis of unsaturated acyl thioesters. In some embodiments, the carbon chain of the acyl group of an acyl thiester has 5, 6, 7, 8, 9, 10, 11, 12, 13, 14, 15, 16, 17, 18, 19, 20, 21, 22, 23, 24, 25, 26, 27 or 28 carbons. In other embodiments, the carbon chain of the acyl group of acyl-thioester is a medium-chain and has 6, 7, 8, 9, 10, 11, 12, 13, 14, 15, 16 or 18 carbons. In other exemplary embodiments the carbon chain of the acyl group of acyl-thioester is 8 carbons in length. In other exemplary embodiments the carbon chain of the acyl group of acyl-thioester is 10 carbons in length. In still other exemplary embodiments, the carbon chain of the acyl group of acyl-thioester is 12 carbons in length. In still other exemplary embodiments, the carbon chain of the acyl group of acyl-thioester is 14 carbons in length. In still other exemplary embodiments, the carbon chain of the acyl group of acyl-thioester is 16 carbons in length. “alkyl-thioesters” are substrates for fatty acid derivative enzymes such as e.g., lactonizing enzymes e.g., YbgC, thioesterases, acyl ACP reductases, ester synthases and their engineered variants that convert the acyl-thioester to fatty acid derivatives such as e.g., natural lactones.

As used herein, the expression “fatty acid derivative biosynthetic pathway” refers to a biochemical pathway that produces fatty acid derivatives. The enzymes that comprise a “fatty acid derivative biosynthetic pathway” are thus referred to herein as “fatty acid derivative biosynthetic polypeptides” or equivalently “fatty acid derivative enzymes”. As discussed supra, the term “fatty acid derivative,” includes a molecule/compound derived from a biochemical pathway that includes a fatty acid derivative enzyme. Thus, a lactonizing enzyme such as e.g., E. coli ybgC, is a “fatty acid derivative biosynthetic peptide” or equivalently a “fatty acid derivative enzyme”. Similarly, a thioesterase enzyme (e.g., an enzyme having thioesterase activity EC 3.2.1.14) is a “fatty acid derivative biosynthetic peptide” or equivalently a “fatty acid derivative enzyme.” Thus the term “fatty acid derivative enzymes” or equivalently “fatty acid derivative biosynthetic polypeptides” refers to, collectively and individually, enzymes that may be expressed or overexpressed to produce fatty acid derivatives such as e.g., natural lactones. Non-limiting examples of “fatty acid derivative enzymes” or equivalently “fatty acid derivative biosynthetic polypeptides” include e.g., fatty acid synthases, lactonizing enzymes, thioesterases, acyl-CoA synthetases, acyl-CoA reductases, acyl ACP reductases, alcohol dehydrogenases, alcohol oxidases, aldehyde dehydrogenases, alcohol O-acyltransferases, fatty alcohol-forming acyl-CoA reductases, fatty acid decarboxylases, fatty aldehyde decarbonylases and/or oxidative deformylases, carboxylic acid reductases, fatty alcohol O-acetyl transferases, hydroxylating enzymes, ester synthases, etc. “Fatty acid derivative enzymes” or equivalently “fatty acid derivative biosynthetic polypeptides” convert substrates into fatty acid derivatives. In exemplary embodiments, a suitable substrate for a fatty acid derivative enzyme is a first fatty acid derivative, e.g. a 5-hydroxy fatty acid, which is converted by the fatty acid derivative enzyme into a different, second fatty acid derivative e.g., a natural δ-lactone.

The term “YbgC protein” or YbgC enzyme” as used herein, refers to a protein/enzyme having lactonizing activity and having at least about 60% sequence identity to SEQ ID NO: 1 over the full length of the protein

The expression “lactonizing activity” as used herein, refers to the ability of an enzyme/protein to convert a hydroxy-fatty acid substrate to a lactone under physiological conditions. Thus, “lactonizing enzymes” have “lactonizing activity”. In an embodiment, a lactonizing enzyme converts a 4-hydroxy fatty acid to a γ-lactone. In an embodiment, a lactonizing enzyme converts a 5-hydroxy fatty acid to a δ-lactone. In an embodiment, a lactonizing enzyme converts a 6-hydroxy fatty acid to an ε-lactone.

In embodiments, “lactonizing activity” is measured by measuring the amount e.g., volume, weight, titer, etc of a substrate e.g., a hydroxyl fatty acid substrate, that is converted to a particular lactone or lactones by a recombinant bacterium that expresses a putative “lactonizing enzyme” and comparing the measured amount to the amount of the same lactone produced by an isogenic bacterial strain that does not express the putative “lactonizing enzyme”.

For example, the amount of hydroxyl fatty acid substrate that is converted to a particular lactone or lactones by a wild type E. coli strain is measured and compared to the amount hydroxyl fatty acid substrate that is converted to a particular lactone or lactones by an isogenic E. coli strain that heterologously expresses an enzyme with potential lactonizing activity. Typically, enzyme with potential lactonizing activity is said to have “lactonizing activity” when the strain that expresses an enzyme with potential lactonizing activity produces an amount of lactone that is at least 3% more than that of the control strain. In some embodiments, a strain expressing an enzyme having “lactonizing activity” produces an amount of lactone that is 5% more, 6% more, 7% more, 8% more, 9% more, 10% more, 20% more, 30% more, 40% more, 50% more, 60% more, 70% more, 75% more, 80% more, 90% more, 95% more, 100% more or more than 100% more than that of the control strain.

Alternatively, if a bacterium expresses an enzyme suspected of having lactonizing activity, then the gene encoding the enzyme with suspected of having lactonizing activity can be deleted and the amount of hydroxyl fatty acid substrate that is converted to a particular lactone or lactones by the strain carrying the deletion is measured and compared to the amount hydroxyl fatty acid substrate that is converted to the particular lactone or lactones by an isogenic strain that does not carry deletion of the enzyme suspected of having lactonizing activity. As above, the deleted enzyme would be said to have “lactonizing activity” when the strain that expresses an enzyme with potential lactonizing activity produces an amount of lactone that is at least 3% more than that of the control strain. In some embodiments, a strain expressing an enzyme having “lactonizing activity” produces an amount of lactone that is 5% more, 6% more, 7% more, 8% more, 9% more, 10% more, 20% more, 30% more, 40% more, 50% more, 60% more, 70% more, 75% more, 80% more, 90% more, 95% more, 100% more or more than 100% more than that of the strain from which the enzyme is deleted.

In some embodiments, “lactonizing activity” is measured by measuring the amount e.g., volume, weight, titer, etc of a substrate e.g., a hydroxyl fatty acid substrate, that is converted to a particular lactone or lactones by a recombinant bacterium that expresses a putative “lactonizing enzyme” and an acyl-CoA synthetase. Laconizing activity is determined as discussed above. The production of the lactone is measured using any convenient method known in the art e.g., using GC-MS. The lactone can be made either in vivo or using an in vitro system.

The expression “physiological conditions” as used herein, refers to typical culture conditions for growing bacteria, e.g., E. coli. Such conditions are well known in the art see e.g., Sambrook et al., and Current Protocols in Molecular Biology Volumes 1-3, John Wiley & Sons, Inc. (1994-1998) and Supplements 1-115 (1987-2016) supra. For example, typically bacterial cultures are grown at neutral pH and at a temperature of between about 25° C. to 42° C., typically at about 37° C. Although bacteria exist which are able to grow under acidic conditions i.e. acidophiles, as used herein, the expression “physiological conditions” specifically excludes acidic conditions and refers particularly to conditions of neutral pH.

The expression “hydroxy acyl-CoA substrate” as used herein refers to a natural lactone precursor which comprises hydroxy fatty acid and coenzyme A linked together through an hydroxy acyl-CoA thioester bond. Typically, a “hydroxy acyl-CoA substrate” is formed by the activity of an acyl-CoA synthetase acting on a hydroxy fatty acid and coenzyme A to form the hydroxy acyl-CoA thioester bond and thereby to produce an hydroxy acyl-CoA molecule.

Sequence Accession numbers throughout this description were obtained from databases provided by the NCBI (National Center for Biotechnology Information) maintained by the National Institutes of Health, U.S.A. (which are identified herein as “NCBI Accession Numbers” or alternatively as “GenBank Accession Numbers” or alternatively a simply “Accession Numbers”), and from the UniProt Knowledgebase (UniProtKB) and Swiss-Prot databases provided by the Swiss Institute of Bioinformatics (which are identified herein as “UniProtKB Accession Numbers”).

The term “enzyme classification (EC) number” refers to a number that denotes a specific polypeptide sequence or enzyme. EC numbers classify enzymes according to the reaction they catalyze. EC numbers are established by the nomenclature committee of the international union of biochemistry and molecular biology (IUBMB), a description of which is available on the IUBMB enzyme nomenclature website on the world wide web.

As used herein, the term “isolated,” with respect to products (such as enzymatically produced lactones as disclosed herein) refers to products that are separated from cellular components, cell culture media, or chemical or synthetic precursors. The enzymatically produced lactones disclosed herein produced by the methods disclosed herein can be relatively immiscible in the fermentation broth, as well as in the cytoplasm. Therefore, in exemplary embodiments, the enzymatically produced lactones disclosed herein collect in an organic phase extracellularly and are thereby “isolated”.

As used herein, the terms “polypeptide” and “protein” are used interchangeably to refer to a polymer of amino acid residues that is typically 12 or more amino acids in length. Polypeptides less than 12 amino acids in length are referred to herein as “peptides”. The terms apply to amino acid polymers in which one or more amino acid residue is an artificial chemical mimetic of a corresponding naturally occurring amino acid, as well as to naturally occurring amino acid polymers and non-naturally occurring amino acid polymers. The term “recombinant polypeptide” refers to a polypeptide that is produced by recombinant techniques, wherein generally DNA or RNA encoding the expressed protein is inserted into a suitable expression vector that is in turn used to transform a host cell to produce the polypeptide. In some exemplary embodiments, DNA or RNA encoding an expressed peptide, polypeptide or protein is inserted into the host chromosome via homologous recombination or other means well known in the art, and is so used to transform a host cell to produce the peptide or polypeptide. Similarly, the terms “recombinant polynucleotide” or “recombinant nucleic acid” or “recombinant DNA” are produced by recombinant techniques that are known to those of skill in the art (see e.g., methods described in Sambrook et al. (supra) and/or Current Protocols in Molecular Biology (supra).

When referring to two nucleotide or polypeptide sequences, the “percentage of sequence identity” between the two sequences is determined by comparing the two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The “percentage of sequence identity” is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

Thus, the expression “percent identity,” or equivalently “percent sequence identity” in the context of two or more nucleic acid sequences or peptides or polypeptides, refers to two or more sequences or subsequences that are the same or have a specified percentage of nucleotides or amino acids that are the same (e.g., about 50% identity, preferably 55%, 60%, 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured e.g., using a BLAST or BLAST 2.0 sequence comparison algorithm with default parameters (see e.g., Altschul et al. (1990) J. Mol. Biol. 215 (3): 403-410) and/or the NCBI web site at ncbi.nlm.nih.gov/BLAST/) or by manual alignment and visual inspection. Percent sequence identity between two nucleic acid or amino acid sequences also can be determined using e.g., the Needleman and Wunsch algorithm that has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6 (Needleman and Wunsch (1970) J. Mol. Biol. 48:444-453). The percent sequence identity between two nucleotide sequences also can be determined using the GAP program in the GCG software package, using a NWSgapdna.CMP matrix and a gap weight of 40, 50, 60, 70, or 80 and a length weight of 1, 2, 3, 4, 5, or 6. One of ordinary skill in the art can perform initial sequence identity calculations and adjust the algorithm parameters accordingly. A set of parameters that may be used if a practitioner is uncertain about which parameters should be applied to determine if a molecule is within a sequence identity limitation of the claims, are a Blossum 62 scoring matrix with a gap penalty of 12, a gap extend penalty of 4, and a frameshift gap penalty of 5. Additional methods of sequence alignment are known in the biotechnology arts (see, e.g., Rosenberg (2005) BMC Bioinformatics 6:278; Altschul et al. (2005) FEBS J. 272 (20): 5101-5109).

Two or more nucleic acid or amino acid sequences are said to be “substantially identical,” when they are aligned and analyzed as discussed above and are found to share about 50% identity, preferably 55%, 60%, 65%, 70%, 75%, 80%, 85%, 90%, 91%, 92%, 93%, 94%, 95%, 96%, 97%, 98%, 99%, or higher identity over a specified region. Two nucleic acid sequences or polypeptide sequences are said to be “identical” if the sequence of nucleotides or amino acid residues, respectively, in the two sequences are the same when aligned for maximum correspondence as described above. This definition also refers to, or may be applied to, the compliment of a test sequence. Identity is typically calculated over a region that is at least about 25 amino acids or nucleotides in length, or more preferably over a region that is 50-100 amino acids or nucleotides in length, or over the entire length of a given sequence.

The expressions “hybridizes under low stringency, medium stringency, high stringency, or very high stringency conditions” describe conditions for hybridization and washing. Guidance for performing hybridization reactions can be found e.g., in Current Protocols in Molecular Biology, John Wiley & Sons, N.Y. (1989), 6.3.1-6.3.6. Aqueous and non-aqueous methods are described in the cited reference and either method can be used. Specific hybridization conditions referred to herein are as follows: (1) low stringency hybridization conditions—6× sodium chloride/sodium citrate (SSC) at about 45° C., followed by two washes in 0.2×SSC, 0.1% SDS at least at 50° C. (the temperature of the washes can be increased to 55° C. for low stringency conditions); (2) medium stringency hybridization conditions—6×SSC at about 45° C., followed by one or more washes in 0.2×SSC, 0.1% SDS at 60° C.; (3) high stringency hybridization conditions—6×SSC at about 45° C., followed by one or more washes in 0.2.X SSC, 0.1% SDS at 65° C.; and (4) very high stringency hybridization conditions—0.5M sodium phosphate, 7% SDS at 65° C., followed by one or more washes at 0.2×SSC, 1% SDS at 65° C. Very high stringency conditions (4) are the preferred conditions unless otherwise specified.

The term “endogenous” as used herein refers to a substance e.g., a nucleic acid, protein, etc. that is produced from within a cell. Thus, an “endogenous” polynucleotide or polypeptide refers to a polynucleotide or polypeptide produced by the cell. In some exemplary embodiments an “endogenous” polypeptide or polynucleotide is encoded by the genome of the parental cell (or host cell). In other exemplary embodiments, an “endogenous” polypeptide or polynucleotide is encoded by an autonomously replicating plasmid carried by the parental cell (or host cell). In some exemplary embodiments, an “endogenous” gene is a gene that was present in the cell when the cell was originally isolated from nature i.e., the gene is “native to the cell”. In other exemplary embodiments, an “endogenous” gene has been altered through recombinant techniques e.g., by altering the relationship of control and coding sequences. Thus, a “heterologous” gene may, in some exemplary embodiments, be “endogenous” to a host cell.

In contrast, an “exogenous” polynucleotide or polypeptide, or other substance (e.g., fatty acid derivative or oleochemical, small molecule compound, etc.) refers to a polynucleotide or polypeptide or other substance that is not produced by the parental cell and which is therefore added to a cell, a cell culture or assay from outside of the cell. Thus, a molecule or compound

As used herein the term “native” refers to the form of a nucleic acid, protein, polypeptide or a fragment thereof that is isolated from nature or a nucleic acid, protein, polypeptide or a fragment thereof that is in its natural state without intentionally introduced mutations in the structural sequence and/or without any engineered changes in expression such as e.g., changing a developmentally regulated gene to a constitutively expressed gene.

As used herein, the term “fragment” of a polypeptide refers to a shorter portion of a full-length polypeptide or protein ranging in size from two amino acid residues to the entire amino acid sequence minus one amino acid residue. In certain embodiments of the disclosure, a fragment refers to the entire amino acid sequence of a domain of a polypeptide or protein (e.g., a substrate binding domain or a catalytic domain).

The term “gene” as used herein, refers to nucleic acid sequences e.g., DNA sequences, which encode either an RNA product or a protein product, as well as operably-linked nucleic acid sequences that affect expression of the RNA or protein product (e.g., expression control sequences such as e.g., promoters, enhancers, ribosome binding sites, translational control sequences, etc). The term “gene product” refers to either the RNA e.g., tRNA, mRNA and/or protein expressed from a particular gene.

The term “expression” or “expressed” as used herein in reference to a gene, refers to the production of one or more transcriptional and/or translational product(s) of a gene. In exemplary embodiments, the level of expression of a DNA molecule in a cell is determined on the basis of either the amount of corresponding mRNA that is present within the cell or the amount of protein encoded by that DNA produced by the cell. The term “expressed genes” refers to genes that are transcribed into messenger RNA (mRNA) and then translated into protein, as well as genes that are transcribed into other types of RNA, such as e.g., transfer RNA (tRNA), ribosomal RNA (rRNA), and regulatory RNA, which are not translated into protein.

The level of expression of a nucleic acid molecule in a cell or cell free system is influenced by “expression control sequences” or equivalently “regulatory sequences”. “Expression control sequences” or “regulatory sequences” are known in the art and include, for example, promoters, enhancers, polyadenylation signals, transcription terminators, nucleotide sequences that affect RNA stability, internal ribosome entry sites (IRES), and the like, that provide for the expression of the polynucleotide sequence in a host cell. In exemplary embodiments, “expression control sequences” interact specifically with cellular proteins involved in transcription (see e.g., Maniatis et al., Science, 236:1237-1245 (1987); Goeddel, Gene Expression Technology: Methods in Enzymology, Vol. 185, Academic Press, San Diego, Calif. (1990)). In exemplary methods, an expression control sequence is operably linked to a polynucleotide sequence. By “operably linked” is meant that a polynucleotide sequence and an expression control sequence(s) are functionally connected so as to permit expression of the polynucleotide sequence when the appropriate molecules (e.g., transcriptional activator proteins) contact the expression control sequence(s). In exemplary embodiments, operably linked promoters are located upstream of the selected polynucleotide sequence in terms of the direction of transcription and translation. In some exemplary embodiments, operably linked enhancers can be located upstream, within, or downstream of the selected polynucleotide.

As used herein, the phrase “the expression of said nucleotide sequence is modified relative to the wild type nucleotide sequence”, refers to a change e.g., an increase or decrease in the level of expression of an native nucleotide sequence or a change e.g., an increase or decrease in the level of the expression of a heterologous or non-native polypeptide-encoding nucleotide sequence as compared to a control nucleotide sequence e.g., wild-type control. In some exemplary embodiments, the phrase “the expression of said nucleotide sequence is modified relative to the wild type nucleotide sequence,” refers to a change in the pattern of expression of a nucleotide sequence as compared to a control pattern of expression e.g., constitutive expression as compared to developmentally timed expression.

In other embodiments, the polypeptide, polynucleotide, or hydrocarbon having an altered level of expression is “attenuated” or has a “decreased level of expression.” As used herein, “attenuate” and “decreasing the level of expression” mean to express or cause to be expressed a polynucleotide, polypeptide, or hydrocarbon in a cell at a lesser concentration than is normally expressed in a corresponding control cell (e.g., wild type cell) under the same conditions.

A polynucleotide or polypeptide can be attenuated using any method known in the art. For example, in some exemplary embodiments, the expression of a gene or polypeptide encoded by the gene is attenuated by mutating the regulatory polynucleotide sequences which control expression of the gene. In other exemplary embodiments, the expression of a gene or polypeptide encoded by the gene is attenuated by overexpressing a repressor protein, or by providing an exogenous regulatory element that activates a repressor protein. In still other exemplary embodiments, DNA- or RNA-based gene silencing methods are used to attenuate the expression of a gene or polynucleotide. In some embodiments, the expression of a gene or polypeptide is completely attenuated, e.g., by deleting all or a portion of the polynucleotide sequence of a gene.

The degree of overexpression or attenuation can be 1.5-fold or more, e.g., 2-fold or more, 3-fold or more, 5-fold or more, 10-fold or more, or 15-fold or more. Alternatively, or in addition, the degree of overexpression or attenuation can be 500-fold or less, e.g., 100-fold or less, 50-fold or less, 25-fold or less, or 20-fold or less. Thus, the degree of overexpression or attenuation can be bounded by any two of the above endpoints. For example, the degree of overexpression or attenuation can be 1.5-500-fold, 2-50-fold, 10-25-fold, or 15-20-fold.

As used herein, “modified activity” or an “altered level of activity” of a protein/polypeptide in a recombinant host cell refers to a difference in one or more characteristics in the activity the protein/polypeptide as compared to the characteristics of an appropriate control protein e.g., the corresponding parent protein or corresponding wild type protein. Thus, in exemplary embodiments, a difference in activity of a protein having “modified activity” as compared to a corresponding control protein is determined by measuring the activity of the modified protein in a recombinant host cell and comparing that to a measure of the same activity of a corresponding control protein in an otherwise isogenic host cell. Modified activities can be the result of, for example, changes in the structure of the protein (e.g., changes to the primary structure, such as e.g., changes to the protein's nucleotide coding sequence that result in changes in substrate specificity, changes in observed kinetic parameters, changes in solubility, etc.); changes in protein stability (e.g., increased or decreased degradation of the protein) etc.

A “control” sample e.g., a “control” nucleotide sequence, a “control” polypeptide sequence, a “control” cell, etc., or value refers to a sample that serves as a reference, usually a known reference, for comparison to a test sample. For example, in an exemplary embodiment, a test sample comprises an enzymatically produced lactone composition produced by a recombinant bacterium that heterologously expresses a YbgC protein as disclosed herein, while the control sample comprises an enzymatically produced lactone composition made by the corresponding or designated bacterium that does not heterologously express a YbgC protein. One of skill will recognize that controls can be designed for assessment of any number of parameters. Furthermore, one of skill in the art will understand which controls are valuable in a given situation and will be able to analyze data based on comparisons to control values.

The term “overexpressed” as used herein, refers to a gene whose expression is elevated in comparison to a “control” level of expression. In exemplary embodiments, “overexpression” of a gene is caused by an elevated rate of transcription as compared to the native transcription rate for that gene. Since “overexpression” is by definition a non-native form of expression of a gene, “a gene that is “overexpressed” is a gene that is “heterologously expressed” (see below). In other exemplary embodiments, overexpression is caused by an elevated rate of translation of the gene compared to the native translation rate for that gene. Methods of testing for overexpression are well known in the art, for example transcribed RNA levels can be assessed using rtPCR and protein levels can be assessed using SDS page gel analysis.

The term “heterologous” as used herein refers to a polypeptide or polynucleotide which is in a non-native state. In the context of a cell and a protein or cell and a polynucleotide the term “heterologous” refers to a polypeptide or a polynucleotide that is not native to the cell in which it is expressed/produced. Thus, a polynucleotide or a polypeptide is “heterologous” to a cell when the polynucleotide and/or the polypeptide and the cell are not found in the same relationship to each other in nature. Therefore, a polynucleotide or polypeptide sequence is “heterologous” to an organism or a second sequence if it originates from a different organism, different cell type, or different species, or, if from the same species, it is modified from its original form. Thus, in an exemplary embodiment, a polynucleotide or polypeptide is “heterologous” when it is not naturally present in a given organism. For example, a polynucleotide sequence that is native to cyanobacteria can be introduced into a host cell of E. coli by recombinant methods, and the polynucleotide from cyanobacteria is then heterologous to the E. coli cell (i.e., the now recombinant E. coli cell).

Similarly, a polynucleotide or polypeptide is “heterologous” when it is modified from its native form or from its relationship with other polynucleotide sequences or is present in a recombinant host cell in a non-native state. Thus, in an exemplary embodiment, a ‘heterologous” polynucleotide or polypeptide comprises two or more subsequences that are not found in the same relationship to each other in nature. For example, a promoter operably linked to a nucleotide coding sequence derived from a species different from that from which the promoter was derived. Alternatively, in another example, if a promoter is operably linked to a nucleotide coding sequence derived from a species that is the same as that from which the promoter was derived, then the operably-linked promoter and coding sequence are “heterologous” if the coding sequence is not naturally associated with the promoter (e.g. a constitutive promoter operably linked to a developmentally regulated coding sequence that is derived from the same species as the promoter). In other exemplary embodiments, a “heterologous” polynucleotide or polypeptide is modified relative to the wild type sequence naturally present in the corresponding wild type host cell, e.g., an intentional modification e.g., an intentional mutation in the sequence of a polynucleotide or polypeptide or a modification in the level of expression of the polynucleotide or polypeptide. Typically, a heterologous nucleic acid or polynucleotide is recombinantly produced.

The term “recombinant” as used herein, refers to a genetically modified polynucleotide, polypeptide, cell, tissue, or organism. When used with reference to a cell, the term “recombinant” indicates that the cell has been modified by the introduction of a heterologous nucleic acid or protein or has been modified by alteration of a native nucleic acid or protein, or that the cell is derived from a cell so modified and that the derived cell comprises the modification. Thus, for example, “recombinant cells” or equivalently “recombinant host cells” may be modified to express genes that are not found within the native (non-recombinant) form of the cell or may be modified to abnormally express native genes e.g., heterologously expressed native genes may be overexpressed, underexpressed or not expressed at all. In exemplary embodiments, a “recombinant cell” or “recombinant host cell” is engineered to heterologously express a heterologous enzyme pathway capable of enzymatically producing a lactone under physiological conditions e.g., without acidification of the culture medium. A recombinant cell can be derived from a microorganism such as a bacterium, a virus or a fungus. However, typically, as used herein a “recombinant cell” is a “recombinant bacterium”.

In exemplary embodiments, a “recombinant host cell” or “recombinant cell” is used to enzymatically producing one or more natural lactones e.g., γ-lactones, δ-lactone and/or ε-lactones under physiological conditions e.g., without acidification of the culture medium including, but not limited to. Therefore, in some exemplary embodiments a “recombinant host cell” is a “production host” or equivalently, a “production host cell”. In some embodiments, the recombinant cell includes one or more polynucleotides, wherein the polynucleotides encode a polypeptides having fatty acid biosynthetic enzyme activity e.g., a thioesterase, a fatty acid hydoxylating enzyme, an acyl-CoA synthetase, wherein the recombinant cell produces an hydroxy acyl-CoA substrate that is converted to a lactone by virtue of the activity of a lactonizing enzyme e.g., a ybgC protein, when cultured in the presence of a (simple) carbon source at neutral pH, under conditions effective to express the polynucleotides.

When used with reference to a polynucleotide, the term “recombinant” indicates that the polynucleotide has been modified by comparison to the native or naturally occurring form of the polynucleotide or has been modified by comparison to a naturally occurring variant of the polynucleotide. In an exemplary embodiment, a recombinant polynucleotide (or a copy or complement of a recombinant polynucleotide) is one that has been manipulated by the hand of man to be different from its naturally occurring form. Thus, in an exemplary embodiment, a recombinant polynucleotide is a mutant form of a native gene or a mutant form of a naturally occurring variant of a native gene wherein the mutation is made by intentional human manipulation e.g., made by saturation mutagenesis using mutagenic oligonucleotides, through the use of UV radiation, mutagenic chemicals, chemical synthesis etc. Such a recombinant polynucleotide might comprise one or more point mutations, deletions and/or insertions relative to the native or naturally occurring variant form of the gene. Similarly, a polynucleotide comprising a promoter operably linked to a second polynucleotide (e.g., a coding sequence) is a “recombinant” polynucleotide. Accordingly, in an embodiment, a “recombinant” polynucleotide comprises a native enzyme under the control of a heterologous or synthetic promoter. Thus, a recombinant polynucleotide comprises polynucleotide combinations that are not found in nature. A recombinant protein (discussed supra) is typically one that is expressed from a recombinant polynucleotide, and recombinant cells, tissues, and organisms are those that comprise recombinant sequences (polynucleotide and/or polypeptide).

The expression “heterologously expresses” or “heterologously expressed” as used herein, refers to a recombinant bacterium that expresses a recombinant polynucleotide or recombinant polypeptide/protein.

A “production host” or equivalently a “production host cell” is a cell used to produce products. As disclosed herein, a “production host” is typically modified to express or overexpress selected genes, or to have attenuated expression of selected genes. Thus, a “production host” or a “production host cell” is a “recombinant host” or equivalently a “recombinant host cell”. As an example, a “production host” heterologously expresses a ybgC protein. As another example, a “production host” heterologously expresses a ybgC protein and heterologously expresses an acyl CoA synthetase.

The term “acetyl-CoA derived compound” refers to any compound or chemical entity (i.e., intermediate or end product) that is made via a biochemical pathway wherein acetyl-CoA functions as intermediate and/or is made upstream of the compound or chemical entity. For example, a acetyl-CoA derived compound may include, but is not limited to, a fatty acid derivative such as, for example, a fatty acid; a fatty ester including, but not limited to a fatty acid methyl ester (FAME) and/or a fatty acid ethyl ester (FAEE); a fatty alcohol; a fatty aldehyde; a fatty amine; an alkane; an olefin or alkene; a hydrocarbon; a 3-hydroxy fatty acid derivative, a bifunctional fatty acid derivative, a non-native monounsaturated fatty acid derivative, an unsaturated fatty acid derivative, a natural lactone, etc.

As used herein, the terms “purify,” “purified,” or “purification” mean the removal or isolation of a molecule from its environment by, for example, isolation or separation. “Substantially purified” molecules are at least about 60% free (e.g., at least about 65% free, at least about 70% free, at least about 75% free, at least about 80% free, at least about 85% free, at least about 90% free, at least about 95% free, at least about 96% free, at least about 97% free, at least about 98% free, at least about 99% free) from other components with which they are associated. As used herein, these terms also refer to the removal of contaminants from a sample. For example, the removal of contaminants can result in an increase in the percentage of enzymatically produced lactones or other compounds in a sample. For example, when an enzymatically produced lactones or other compound is produced in a recombinant host cell, the enzymatically produced lactone or other compound can be purified by the removal of host cell proteins. After purification, the percentage of enzymatically produced lactone or other compounds in the sample is increased. The terms “purify,” “purified,” and “purification” are relative terms which do not require absolute purity. Thus, for example, when an enzymatically produced lactone is produced in recombinant host cells, an enzymatically produced lactones that is substantially separated from other cellular components (e.g., nucleic acids, polypeptides, lipids, carbohydrates, or other hydrocarbons).

As used herein, the term “attenuate” means to weaken, reduce, or diminish. For example, a polypeptide can be attenuated by modifying the polypeptide to reduce its activity (e.g., by modifying a nucleotide sequence that encodes the polypeptide).

As used herein, the term “carbon source” refers to a substrate or compound suitable to be used as a source of carbon for prokaryotic or simple eukaryotic cell growth. Carbon sources can be in various forms, including, but not limited to polymers, carbohydrates, acids, alcohols, aldehydes, ketones, amino acids, peptides, and gases (e.g., CO and CO₂). Exemplary carbon sources include, but are not limited to, monosaccharides, such as glucose, fructose, mannose, galactose, xylose, and arabinose; oligosaccharides, such as fructo-oligosaccharide and galacto-oligosaccharide; polysaccharides such as starch, cellulose, pectin, and xylan; disaccharides, such as sucrose, maltose, cellobiose, and turanose; cellulosic material and variants such as hemicelluloses, methyl cellulose and sodium carboxymethyl cellulose; succinate, lactate, and acetate; alcohols, such as ethanol, methanol, and glycerol, or mixtures thereof. The carbon source can also be a product of photosynthesis, such as glucose. In certain embodiments, the carbon source is biomass. In other embodiments, the carbon source is glucose. In other embodiments the carbon source is sucrose. In other embodiments the carbon source is glycerol. In other embodiments, the carbon source is a simple carbon source such as e.g., glucose. In other embodiments, the carbon source is a renewable carbon source. In other embodiment, the carbon source is natural gas. In other embodiments the carbon source comprises one or more components of natural gas, such as methane, ethane, or propane. In other embodiments, the carbon source is flu gas or synthesis gas. In still other embodiments, the carbon source comprises one or more components of flu or synthesis gas such as carbon monoxide, carbon dioxide, hydrogen, etc. As used herein, the term “carbon source” or “simple carbon source” specifically excludes oleochemicals such as e.g., saturated or unsaturated fatty acids.

As used herein, the term “biomass” refers to any biological material from which a carbon source is derived. In some embodiments, a biomass is processed into a carbon source which is suitable for bioconversion. In other embodiments, the biomass does not require further processing into a carbon source. The carbon source can be converted into a composition comprising enzymatically produced lactones.

An exemplary source of biomass is plant matter or vegetation, such as corn, sugar cane, or switchgrass. Another exemplary source of biomass is metabolic waste products, such as animal matter (e.g., cow manure). Further exemplary sources of biomass include algae and other marine plants. Biomass also includes waste products from industry, agriculture, forestry, and households, including, but not limited to, glycerol, fermentation waste, ensilage, straw, lumber, sewage, garbage, cellulosic urban waste, and food leftovers (e.g., soaps, oils and fatty acids). The term “biomass” also can refer to sources of carbon, such as carbohydrates (e.g., monosaccharides, disaccharides, or polysaccharides).

### I. Introduction

As discussed above, there is an increasing demand for natural flavor and fragrance ingredients to satisfy the needs of e.g., the food, beverage, fragrance and cosmetics industries.

Typically, flavors and fragrance molecules are extracted from their natural sources, or are prepared using biotechnological processes that involve a chemical conversion step. The extraction of flavors and fragrances from natural sources is laborious process and unfortunately, because many known biotechnological processes require a chemical conversion step, in many countries flavors and fragrances produced by biotechnological processes are disqualified from being marketed and sold as natural flavor (see e.g., EFFA Guidance Document for the Production of Natural Flavouring Substances and (Natural) Flavouring Preparations in the EU).

Therefore, to meet the needs of industry, new methods for the production of natural flavors and fragrances, e.g. lactones, are needed.

Accordingly, the present disclosure provides methods for the biological/enzymatic production of natural lactones without a chemical conversion step. As will be disclosed in detail below, the disclosure provides methods for the enzymatic production of lactones under physiological conditions via a hydroxyacyl-CoA intermediate, in recombinant cells (in vivo) or cell lysates (in vitro).

II. Bacteria that Enzymatically Produce Lactones

### A. General Methods

This disclosure utilizes routine techniques in the field of recombinant genetics. Basic texts disclosing the general methods and terms in molecular biology and genetics include e.g., Sambrook et al., Molecular Cloning, a Laboratory Manual, Cold Spring Harbor Press 4th edition (Cold Spring Harbor, N.Y. 2012); Current Protocols in Molecular Biology Volumes 1-3, John Wiley & Sons, Inc. (1994-1998). This disclosure also utilizes routine techniques in the field of biochemistry. Basic texts disclosing the general methods and terms in biochemistry include e.g., Lehninger Principles of Biochemistry sixth edition, David L. Nelson and Michael M. Cox eds. W. H. Freeman (2012). This disclosure also utilizes routine techniques in industrial fermentation. Basic texts disclosing the general methods and terms in fermentation include e.g., Principles of Fermentation Technology, 3rd Edition by Peter F. Stanbury, Allan Whitaker and Stephen J. Hall. Butterworth-Heinemann (2016); Fermentation Microbiology and Biotechnology, 2nd Edition, E. M. T. El-Mansi, C. F. A. Bryce, Arnold L. Demain and A. R. Allman eds. CRC Press (2007). This disclosure also utilizes routine techniques in the field of organic chemistry. Basic texts disclosing the general methods and terms in organic chemistry include e.g., Practical Synthetic Organic Chemistry: Reactions, Principles, and Techniques, Stephane Caron ed., John Wiley and Sons Inc. (2011); The Synthetic Organic Chemist's Companion, Michael C. Pirrung, John Wiley and Sons Inc. (2007); Organic Chemistry, 9th Edition-Francis Carey and Robert Giuliano, McGraw Hill (2013).

For nucleic acids, sizes are given in either kilobases (kb) or base pairs (bp). Estimates are typically derived from agarose or acrylamide gel electrophoresis, from sequenced nucleic acids, or from published DNA sequences. For proteins, sizes are given in kilodaltons (kDa) or amino acid residue numbers. Protein sizes may be estimated from gel electrophoresis, from sequenced proteins, from derived amino acid sequences, or from published protein sequences.

Oligonucleotides that are not commercially available can be chemically synthesized e.g., according to the solid phase phosphoramidite triester method first described by Beaucage & Caruthers, Tetrahedron Letts. 22:1859-1862 (1981), using an automated synthesizer, as described in Van Devanter et al., Nucleic Acids Res. 12:6159-6168 (1984). Purification of oligonucleotides is e.g., by either native acrylamide gel electrophoresis or by anion-exchange HPLC as described in Pearson & Reanier, J. Chrom. 255:137-149 (1983).

The sequence of cloned genes and synthetic oligonucleotides can be verified after cloning using, e.g., the chain termination method for sequencing double-stranded templates of Wallace et al., Gene 16:21-26 (1981).

B. E. coli YbgC Thioesterase and Related YbgC-Like Thioesterase Enzymes

### 1. YbgC Protein Structure and Lactonizing Activity

The sequence of wild type E. coli ybgC is provided below as SEQ ID NO:1. The E. coli ybgC protein also has Uniprot Accession No.: POA8Z3

(SEQ ID NO: 1)
MNTTLFRWPVRVYYEDTDAGGVVYHASYVAFYERARTEMLRHHHFSQQA
LMAERVAFVVRKMTVEYYAPARLDDMLEIQTEITSMRGTSLVFTQRIVN
AENTLLNEAEVLVVCVDPLKMKPRALPKSIVAEFKQ

The wild type E. coli ybgC thioesterase SEQ ID NO:1, is generally accepted to function as a acyl-CoA thioestarase (see e.g., Kuznetsova E., et al.; FEMS Microbiol Rev. 2005 April; 29 (2): 263-79).

E. coli ybgC is a 134 amino acid protein that is known to function as a thioesterase (E.C 3.1.2.-). The linear protein sequence has been analyzed and the 3-dimensional structure of E. coli ybgC has been inferred from sequence information (see e.g., Angelini, A., et al. (2008) Proteins 72:1212-1221; PDB ID code 1S5U).

As disclosed in detail herein, it is shown that in addition to functioning as thioesterases, the family of ybgC enzymes such as e.g., E. coli ybgC, also function in the production of lactones. Thus, as disclosed herein, ybgC enzymes can be used in methods for the preparation of lactones. The role of ybgC in the production of lactones has not been appreciated before.

The linear ybgC protein comprises five (5) beta-strand structures (residues 6-11 (1), 21-23 (β1′), 56-66 (β2), 75-85 (3), 87-97 (4) and 103-115 ((5)) and four (4) alpha helical structures (residues 14-16 (α1), 25-42 (α2), 47-52 (α2′) and 126-131 (α3)). The monomeric protein folds into a classic hot-dog fold structure wherein the long α2 helix (residues 25-42) is surrounded by four antiparallel β-sheets (β2-β5)) see e.g., Angelini, A., et al. (2008) supra.

The active site residues include Y14, D18, H25, F57 and V58 (see e.g., Angelini, A., et al. (2008) supra). As shown in Example 7 herein below, mutation of the active site residue D18 to A18 (D18A) severely reduces lactone forming activity of the ybgC enzyme. Similarly, mutations at Y14, H25, F57 and V58 to non-conservative amino acid(s) are expected to severely reduce or eliminate lactone forming activity.

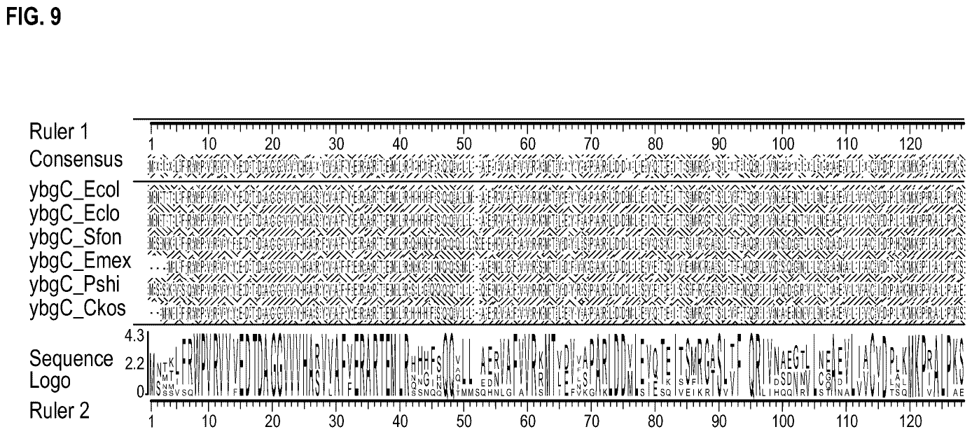

Some residues of ybgC proteins are highly conserved. Non-conservative substitution mutations at these residues are likely to reduce or abolish lactone forming activity. For example, with reference to the E. coli sequence, SEQ ID NO:1 and as shown in FIG. 9, the amino acids corresponding to R11, V12, Y13, Y14, E15, D16, T17, D18, G21, V22, V23, Y24, H25, A26, Y28, V29, F31, E33, R34, A35, R36, T37, E38, F57, V58, V59, Y66, A70, L72, L76, 178, M85, R86, L90, Q94, 196, L104, V109, V112, L124 are highly conserved among the ybgC proteins. Therefore, non-conservative substitution mutations or deletion mutations at these sites are likely to have an effect on lactone forming activity and may reduce lactone forming activity significantly.

Other functional regions include regions having helical domains such as the region encompassing the α2′ helical region which is thought to form part of the substrate binding pocket V58 (see e.g., Angelini, A., et al. (2008) supra). Mutations in this region which affect the helical structure or alter the charge profile may affect substrate specificity but are not expected to eliminate lactone forming activity.

Furthermore generally, as to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Such “conservatively modified variants” are likely to have minimal to no effect on protein function especially if they occur in regions that are less highly conserved and are outside of the active site and outside of the regions of helical and/or beta-strand structures.

Conservative substitution tables providing functionally similar amino acids are well known in the art. Such conservatively modified variants are in addition to and do not exclude polymorphic variants, interspecies homologs, and alleles of the invention. The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine(S), Threonine (T); and 8) Cysteine (C), Methionine (M) (see, e.g., Creighton, Thomas E. (1992) Proteins: Structures and Molecular Properties).

Thus, in embodiments, the disclosure provides recombinant bacteria that heterologously express a ybgC protein which are useful in methods for the enzymatic production of lactones e.g., γ-, δ- or ε-lactones, under physiological conditions without the need for a chemical conversion step.

### 2. Assaying for Lactonizing Activity of ybgC and Related ybgC-Like Proteins

In embodiments, lactonizing activity of ybgC and ybgC-like proteins is measured as disclosed e.g., in Examples 1-7 herein below.

In some embodiments, lactonizing activity of ybgC and ybgC-like proteins is measured by measuring the titer of γ-, δ- or ε-lactones produced by a bacterial strain comprising a heterologously expressed ybgC protein (i.e., a test strain) and comparing that value to the titer of the corresponding lactones produced by an appropriate control strain that is isogenic to the test strain except for the YbgC protein that it comprises. Recombinant bacterial strains comprising a heterologously expressed YbgC protein will produce more γ-, δ- or ε-lactones than the control strain when the strains are cultured at a neutral pH.

In some embodiments, the total titer of γ-, δ- or ε-lactones produced are measured and compared between the test and the control strain. In other embodiments, the percent of the total titer of γ-, δ- or ε-lactone produced by a test strain is measured and compared to the percent of the total titer of γ-, δ- or ε-lactone produced by an appropriate control strain that is isogenic to the test strain except for the heterologously expressed YbgC protein.

In exemplary embodiments, Gas-Chromatography with Flame-Ionization Detection (GC-FID) is used to assay the γ-, δ- or ε-lactone. GC-FID is known in the art (see e.g., Adlard, E. R.; Handley, Alan J. (2001). Gas chromatographic techniques and applications. London: Sheffield Academic). However, any appropriate method for quantitation and analysis may be used e.g., mass spectrometry (MS), Gas Chromatography-mass spectrometry (GC-MS), liquid chromatography-mass spectrometry (LC-MS), thin layer chromatography (TLC), etc.

The lactone can be confirmed e.g., either by using authentic standards or by GC/MS of their dimethyl disulphide (DMDS) adducts (see e.g., Nichols et al. 1986, J. Microbiol. Methods 5:49-55).

### 3. Bacteria that Enzymatically Produce δ, γ and ε-Lactones

In an exemplary embodiment, the disclosure provides recombinant bacteria that enzymatically produce δ, γ and ε-lactones under physiological conditions e.g., at neutral pH and without raising the temperature of the culture.

In embodiments, an enzymatic pathway for biological/enzymatic production of lactones as disclosed herein comprises an acyl-CoA synthetase (also known as acyl-CoA synthase or acyl-CoA ligase (EC 6.2.1.3)) and a lactone forming or lactonizing enzyme/ybgC protein such as e.g., ybgC from Escherichia coli (SEQ ID NO:1).

One example of an acyl-CoA synthetase suitable for use in a recombinant bacterium for the enzymatic production of lactones is e.g., SEQ ID NO:10 (Uniprot accession Q88PT5), herein referred to as fadD3, from Pseudomonas putida. Other examples of suitable acyl-CoA synthetases include, but are not limited to acyl-CoA synthetases provided below in Table 1.

TABLE 1
Examples of acyl-CoA synthetases EC 6.2.1.3

| | Uniprot | |
|---|---|---|
| Gene | Accession | Organism |
| fadD | P69451 | Escherichia coli |
| fadD-I | Q88EB7 | Pseudomonas putida |
| fadD-II | Q88EB6 | Pseudomonas putida |
| PP_0763 “fadD3” | Q88PT5 | Pseudomonas putida |
| fatty acid-CoA ligase | A0A069Q372 | Pseudomonas aeruginosa |
| fatty acid-CoA ligase | A0A1I1GPT1 | Pseudomonas citronellolis |
| fatty acid-CoA ligase | A4XYJ3 | Pseudomonas mendocina |
| IcfB | O07610 | Bacillus subtilis |
| FAA1 | P30624 | Saccharomyces cerevisiae |

Examples of YbgC proteins (lactonizing enzymes) include, but are not limited to YbgC proteins provided in Table 2 and an alignment of these protein sequences is shown in FIG. 9. Typically, YbgC proteins suitable for use in the methods disclosed herein have at at least 60% sequence identity to SEQ ID NO:1 and have lactonizing activity.

TABLE 2
Examples of ybgC homologs (YbgC proteins)

| | | | Conserved |
|---|---|---|---|
| | | | amino acids/ |
| | Uniprot | | sequence |
| Gene | Accession | Organism | identity |
| ybgC | (P0A8Z3) | Escherichia coli | 100% |
| ybgC | A0A078LJN7 | Citrobacter koseri | 93% |
| ybgC | A0A157YV01 | Enterobacter cloacae | 94% |
| Acyl-CoA | A0A0F7D2B7 | Serratia fonticola | 71% |
| thioesterase | | | |
| Acyl-CoA | A0A099DCT9 | Exiguobacterium | 68% |
| thioesterase | | mexicanum | |
| ybgC | A0A1A9AWY4 | Plesiomonas | 62% |
| | | shigelloides | |

A recombinant microbe that heterologously expresses a biological pathway comprising a heterologously expressed ybgC protein and a heterologously expressed acyl-CoA synthetase converts 4-hydroxy fatty acids, e.g. 4-hydroxydecanoic acid, 5-hydroxy fatty acids, e.g. 5-hydroxydecanoic acid, and 6-hydroxy fatty acids, e.g. 6-hydroxyhexanoic acid, to the corresponding γ-, δ-, and ε-lactones, respectively, without the need of a chemical step for lactonization, e.g. a chemical step characterized by lowering the pH of the fermentation broth and raising the temperature of the culture.

The 4-, 5-, or 6-hydroxy fatty acid can be provided to the recombinant microbe by (i) adding the 4-, 5-, or 6-hydroxy fatty acid directly to the fermentation medium or (ii) by adding an oleochemical that contains a longer chain hydroxylated fatty acid such as e.g., ricinoleic acid to the fermentation medium or (iii) by having the recombinant microbe endogenously producing 4-, 5-, or 6-hydroxy fatty acids from a simple carbon source.

### 1. 4-, 5-, or 6-Hydroxy Fatty Acid as Substrates:

In embodiments, the disclosure provides methods for enzymatically producing a lactone under physiological conditions. The method comprises culturing a recombinant bacterium that heterologously expresses a YbgC protein and an acyl-CoA synthetase protein, in a culture medium having a neutral pH. To prepare lactones, the culture of recombinant bacteria is fed an exogenous source of hydroxyl-fatty acids. In one embodiment, the culture of recombinant bacteria is fed an exogenous 4-hydroxy fatty acid and as a result produces a γ-lactone. In one embodiment, the culture of recombinant bacteria is fed an exogenous 5-hydroxy fatty acid and as a result produces a δ-lactone. In one embodiment, the culture of recombinant bacteria is fed an exogenous 6-hydroxy fatty acid and as a result produces an ε-lactone.

In one embodiment, recombinant microbe heterologously expressing an acyl-CoA synthetase, e.g. FadD3 from P. putida, and a lactonizing enzyme, e.g. ybgC from E. coli, converts exogenously added 4-hydroxydodecanoic acid to γ-dodecalactone without acidification.

In one embodiment, a recombinant microbe heterologously expressing an acyl-CoA synthetase, e.g. FadD3 from P. putida, and a lactonizing enzyme, e.g. ybgC from E. coli, converts exogenously added 4-hydroxydecanoic acid to γ-decalactone without acidification.

In one embodiment, a recombinant microbe heterologously expressing an acyl-CoA synthetase, e.g. FadD3 from P. putida, and a lactonizing enzyme, e.g. ybgC from E. coli, converts exogenously added 4-hydroxyoctanoic acid to γ-octalactone without acidification.

In one embodiment, a recombinant microbe heterologously expressing an acyl-CoA synthetase, e.g. FadD3 from P. putida, and a lactonizing enzyme, e.g. ybgC from E. coli, converts exogenously added 4-hydroxytetradecanoic acid to γ-tetradecalactone without acidification.

In one embodiment, a recombinant microbe heterologously expressing an acyl-CoA synthetase, e.g. FadD3 from P. putida, and a lactonizing enzyme, e.g. ybgC from E. coli, converts exogenously added 5-hydroxydecanoic acid to δ-decanolactone without acidification.

In one embodiment, a recombinant microbe heterologously expressing an acyl-CoA synthetase, e.g. FadD3 from P. putida, and a lactonizing enzyme, e.g. ybgC from E. coli, converts exogenously added 6-hydroxydecanoic acid to δ-decanolactone without acidification.

In one embodiment, a recombinant microbe heterologously expressing an acyl-CoA synthetase, e.g. FadD3 from P. putida, and a lactonizing enzyme, e.g. ybgC from E. coli, converts exogenously added 6-hydroxyhexanoic acid to ε-caprolactone without acidification.

### 1. Oleochemicals as Substrates:

In embodiments, the disclosure provides methods for enzymatically producing a lactone under physiological conditions. The method comprises culturing a recombinant bacterium that heterologously expresses a YbgC protein and an acyl-CoA synthetase protein, in a culture medium having a neutral pH. To prepare lactones, the culture of recombinant bacteria is fed an exogenous source oleochemicals such as e.g., castor oil, ricinoleic acid ((9z)-12-hydroxy-9-octadecenoic acid), coriolic acid ((9z, Ile)-13-hydroxy-9,11-octadecadienoic acid), lesquerolic acid ((11z)-14-hydroxy-11-icosenoic acid), 11-hydroxy palmitic acid, 10-hydroxy stearic acid or 12-hydroxy stearic acid, via fatty acyl chain shortening by β-oxidation. β-oxidation is well known in the art (see e.g., U.S. Pat. No. 9,017,984). β-oxidation of the recombinant microbe may or may not be attenuated or otherwise altered. Except for ricinoleic acid, which is a major component of the oil of castor beans, all other hydroxy fatty acids are not abundant in nature.

Therefore, in an embodiment, a recombinant bacterium is constructed which heterologously expresses a YbgC protein and an acyl-CoA synthetase protein and which also bears either an attenuated or deleted endogenous acyl-CoA dehydrogenase (EC 1.3.99.3). Optionally in an embodiment, a heterologous acyl-CoA dehydrogenase with low activity towards medium-chain acyl-CoAs, e.g. PpACX1 from Prunus persica (see e.g., Zhang et al., Plant Cell Rep (2017) 36:829-842) or Aox2 from Yarrowia lipolytica (see e.g., Wache et al., Appl Microbiol Biotechnol (2003) 61:393-404) can be expressed in place of attenuated or deleted endogenous acyl-CoA dehydrogenase (EC 1.3.99.3)

In an embodiment, a recombinant bacterium is constructed which heterologously expresses a YbgC protein (e.g., SEQ ID NO:1) and an acyl-CoA synthetase protein (e.g., FadD3 from P. putida) and also has the acyl-CoA dehydrogenase (fadE, EC 1.3.99.3) deleted, the acetyl-CoA C-acyltransferase (also known as acyl-CoA thiolase, fadA, EC 2.3.1.16) and 3-ketoacyl-CoA reductase/3-hydroxyacyl-CoA dehydrogenase (fadB, 1.1.1.35 3) constitutively expressed or overexpressed and a long-chain acyl-CoA dehydrogenase e.g., Aox2 from Y. lipolytica overexpressed, converts exogenously added ricinoleic acid or 12-hydroxystearic to γ-decalactone without acidification.

In an embodiment, a recombinant bacterium is constructed which heterologously expresses a YbgC protein (e.g., SEQ ID NO:1) and an acyl-CoA synthetase protein (e.g., FadD3 from P. putida). The recombinant bacterium further has the acyl-CoA dehydrogenase (fadE, EC 1.3.99.3) deleted, the acetyl-CoA C-acyltransferase (also known as acyl-CoA thiolase) (fadA, EC 2.3.1.16) and 3-ketoacyl-CoA reductase/3-hydroxyacyl-CoA dehydrogenase (fadB, 1.1.1.35 3) is either constitutively expressed or over expressed and a long-chain acyl-CoA dehydrogenase such as e.g., Aox2 from Y. lipolytica overexpressed, converts exogenously added 10-hydroxystearic acid to Y-dodecalactone without acidification.

### 3. Simple Carbon Source as Substrate

In further embodiments, the disclosure provides methods for enzymatically producing a lactone under physiological conditions from a simple carbon source. In these embodiments, fatty acid and/or oleochemical feedstocks are not required, as the disclosed recombinant bacteria use simple carbon sources to produce hydroxyl acyl-CoA substrates that are converted to lactones by the recombinant bacterium.

Therefore, the disclosure provides methods for enzymatically producing a lactone under physiological conditions from a simple carbon source wherein the method comprises culturing a recombinant bacterium that heterologously expresses a lactonizing enzyme (e.g., a YbgC protein e.g., SEQ ID NO:1) and an acyl-CoA synthetase protein (e.g., FadD3 from P. putida). The recombinant bacterium further comprises a heterologously expressed thioesterase and a fatty acid-hydroxylating enzyme (see e.g., WO 2014/201474 A1), in a culture medium having a neutral pH.

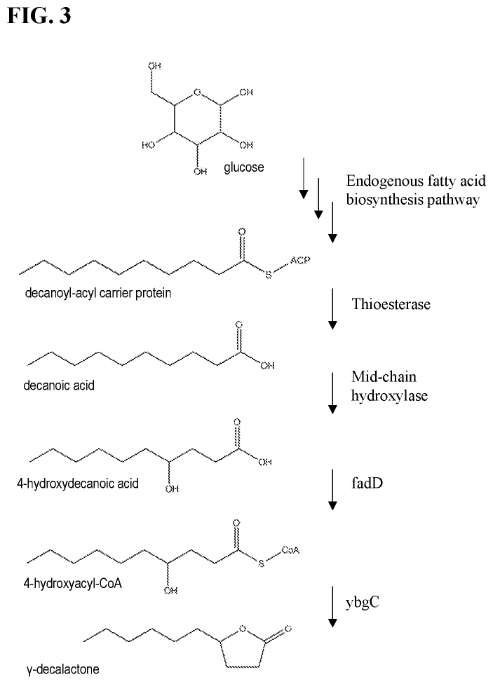

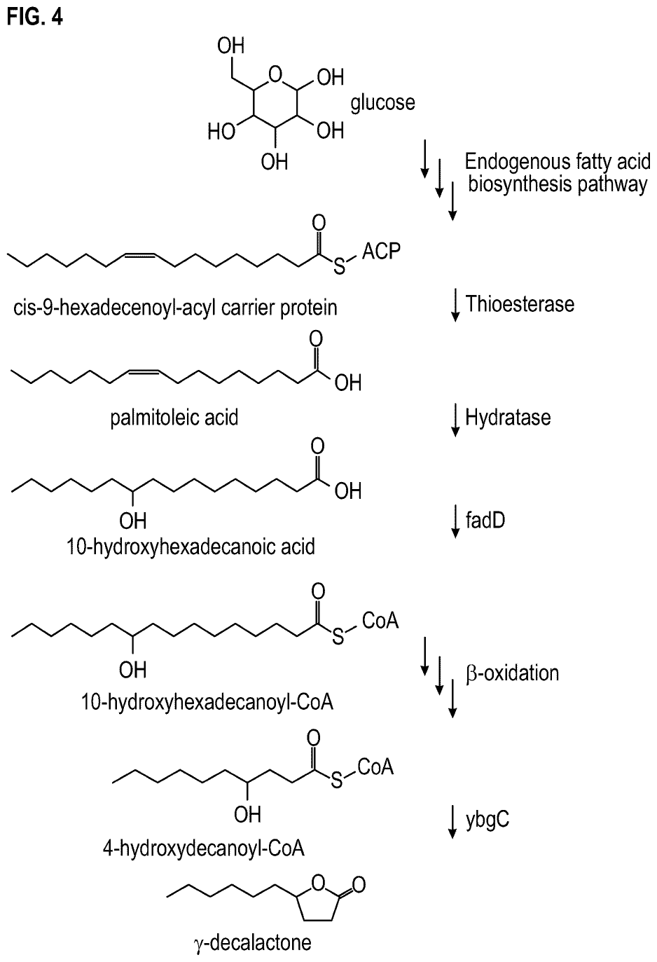

A recombinant bacterium that comprises a heterologously expressed lactonizing enzyme (e.g., YbgC protein, e.g., SEQ ID NO:1) and an acyl-CoA synthetase protein (e.g., FadD3 from P. putida) and which heterologously expresses thioesterase and a heterologously expressed fatty acid-hydroxylating enzyme, produces 4-, 5-, or 6-hydroxy fatty acids endogenously from a simple carbon source such as a carbohydrate, e.g. glucose, via an activated acyl-thioester of a medium-chain fatty acid intermediate, e.g. decanoic acid or dodecanoic acid (see e.g., FIG. 3), or via an activated acyl-thioester of a long-chain fatty acid intermediate, e.g. plamitoleic acid or oleic acid (see e.g., FIG. 4).

Thus, a biochemical pathway for the production of γ- or δ-lactones that produces the lactones from a simple carbon source via an activated medium-chain acyl-thioester, e.g. acyl-ACP, contains the following enzymes: a thioesterase, a fatty acid-hydroxylating enzyme, an acyl-CoA synthetase such as e.g., FadD3 from P. putida and a lactonizing enzyme such as ybgC from E. coli. Examples of suitable thioesterases include but are not limited to those thioesterase enzymes provided in Table 3.

TABLE 3
Examples of thioesterases EC 3.1.2.

| | Uniprot/GenBank | |
|---|---|---|
| Gene | Accession | Organism |
| FatB1 | Q42561 | Umbellularia california |
| FatB1 | Q39473 | Cinnamomum camphorum |
| FatB1 | NP_001310677 | Ricinus communis |
| FatB2 | Q39514 | Cuphea hookeriana |
| FatB2 | Q39555 | Cuphea palustris |
| FatB3 | CAB60830 | Cuphea lanceolata |
| FatA | NP_189147 | Arabidopsis thaliana |
| FatA | XP_021976166 | Helianthus annuus |
| FatA1 | ACB29661 | Macadamia tetraphylla |

Examples of suitable hydroxylating enzymes include but are not limited to those hydroxylases provided in Table 4. Hydroxylase enzymes suitable for hydroxylating e.g. decanoic acid to 4-hydroxy decanoic acid, can be isolated from microorganisms such as Mucor circinelloides, Umbellularia isabellina or Aspergillus oryzae (see e.g., U.S. Pat. Nos. 5,457,036 and 7,863,023). Such hydroxylases can be isolated by the following methods: Potential hydroxylases, e.g. cytochrome P450 oxygenases, can be identified from the genome sequences of these microorganisms based on their DNA sequence. The identified hydroxylase genes can be amplified from cDNA and cloned in a bacterial or fungal expression vector. These expression constructs can be transformed in a bacterial, e.g. Escherichia coli, or fungal host, e.g. Saccharomyces cerevisiae, and the resulting cells or cell extracts can be evaluated in an in-vivo or in-vitro hydroxylase assay that is able to detect the enzymatic conversion of e.g. decanoic acid to 4-hydroxy decanoic acid. Alternatively, or if a genome sequence is unknown, a cDNA expression library can be constructed in a bacterial or fungal expression vector and transformed into in a bacterial, e.g. Escherichia coli, or fungal host, e.g. Saccharomyces cerevisiae, and the resulting cells or cell extracts can be screened in an in vivo or in vitro hydroxylase assay that is able to detect the enzymatic conversion of e.g. decanoic acid to 4-hydroxy decanoic acid.

TABLE 4
P450 hydroxylases suitable for hydroxylating
dodecanoic acid to 5-hydroxy dodecanoic acid

| | | GenBank | |
|---|---|---|---|
| Name | microorganism | accession | reference |
| CYP505E3 | Aspergillus | XP_001210151 | WO2017/137935 |
| | terreus | | |
| CYP505Ak | Aspergillus | GAA88365 | WO2017/137935 |
| | kawachii | | |
| CYP505E1 | Aspergillus niger | CAK39220 | WO2017/137935 |
| CYP505Pe | Penicillium | XP_016592781 | WO2017/137935 |
| | expansum | | |
| CYP505An | Aspergillus niger | GAQ43769 | WO2017/137935 |
| CYP505Pc | Penicillium | CRL20473 | WO2017/137935 |
| | camemberti | | |
| CYP505Pf | Penicillium freii | KUM63529 | WO2017/137935 |
| CYP102A1 | Bacillus | AAA87602 | Dietrich, Journal |
| variants | megaterium | | of Biotechnology |
| | | | 139 (2009) 115-117 |

Therefore, in an embodiment, a method for producing γ-lactones, from a simple carbon source under physiological conditions comprises culturing a recombinant bacterium that heterologously expresses the following proteins/enzymes: a thioesterase, e.g. FatB1 from Umbellularia californica, a fatty acid hydroxylase, from e.g. Mucor circinelloides, Umbellularia isabellina or Aspergillus oryzae, an acyl-CoA synthetase, e.g. FadD3 from P. putida, and a lactonizing enzyme, e.g. ybgC from E. coli, produces γ-dodecalactone from a simple carbon source without acidification of the culture medium.

In an embodiment, a method for producing γ-lactones from a simple carbon source under physiological conditions comprises culturing a recombinant bacterium that heterologously expresses the following proteins/enzymes: a thioesterase, e.g. FatB3 from Cuphea lanceolata, a fatty acid hydroxylase, e.g. from Mucor circinelloides, Umbellularia isabellina or Aspergillus oryzae, an acyl-CoA synthetase, e.g. FadD3 from P. putida, and a lactonizing enzyme, e.g. ybgC from E. coli, produces γ-decalactone from a simple carbon source without acidification.

In an embodiment, a method for producing γ-lactones from a simple carbon source under physiological conditions comprises culturing a recombinant bacterium that heterologously expresses the following proteins/enzymes: a thioesterase, e.g. FatB2 from Cuphea hookeriana, a fatty acid hydroxylase, e.g. from Mucor circinelloides, Umbellularia isabellina or Aspergillus oryzae, an acyl-CoA synthetase, e.g. FadD3 from P. putida, and a lactonizing enzyme, e.g. ybgC from E. coli, produces γ-octalactone from a simple carbon source without acidification.

In an embodiment, a method for producing δ-lactones from a simple carbon source under physiological conditions comprises culturing a recombinant bacterium that heterologously expresses the following proteins/enzymes: a thioesterase, e.g. FatB1 from Umbellularia californica, a fatty acid hydroxylase, see e.g., Table 4, an acyl-CoA synthetase, e.g. FadD3 from P. putida, and a lactonizing enzyme, e.g. ybgC from E. coli, produces δ-dodecalactone from a simple carbon source without acidification.

In an embodiment a recombinant bacterium having the endogenous acyl-CoA dehydrogenase gene (fadE, EC 1.3.99.3) deleted, the acetyl-CoA C-acyltransferase (also known as acyl-CoA thiolase) (fadA, EC 2.3.1.16) and 3-ketoacyl-CoA reductase/3-hydroxyacyl-CoA dehydrogenase (fadB, 1.1.1.35 3) either constitutively expressed or over expressed and a long-chain acyl-CoA dehydrogenase such as Aox2 from Y. lipolytica overexpressed, and with the expression a thioesterase, e.g. FatA3 from Arabidopsis thaliana, a fatty acid hydratase, e.g. OhyA from Stenotrophomonas maltophilia, an acyl-CoA synthetase, e.g. FadD3 from P. putida, and a lactonizing enzyme, e.g. ybgC from E. coli, produces-dodecalactone and-decalactone and from a carbohydrate without acidification.

In an embodiment, biochemical pathways that produce lactones from a simple carbon source in microbial/bacterial strains are expressed in strains that synthesize odd-chain fatty acid (see e.g., U.S. Pat. No. 8,372,610), and the corresponding odd-chain lactones are produced, e.g. natural γ- or δ-nonalactone, γ- or δ-undecalactone, γ- or δ-tridecalactone, etc.

In another embodiment biochemical pathways that produce lactones from a simple carbon source in microbial/bacterial strains that produce branched-chain fatty acid (see e.g., U.S. Pat. No. 8,530,221), the corresponding branched-chain lactones are produced, e.g. natural 9-methyl-γ- or δ-decalactone, 8-methyl-γ- or δ-decalactone, 10-methyl-γ- or δ-undecalactone, 11-methyl-γ- or δ-dodecalactone or 10-methyl-γ- or δ-dodecalactone, etc.

In another embodiment biochemical pathways that produce lactones from a simple carbon source in microbial/bacterial strains that produce ω-7 monounsaturated fatty acid derivatives monounsaturated lactones are formed originating from the ω7-monounsaturated acyl-fatty acids, e.g. natural (5z)-dodeceno-γ- or δ-lactone, (7z)-tetradeceno-γ- or δ-lactone, etc.

The δ- or γ-lactones produced by this method are chiral molecules. They include all chiral, diastereomeric, and racemic forms of the molecules, including enriched or resolved optical isomers, e.g. (S)-γ-decanolactone, (R)-γ-decanolactone, (S)-8-decanolactone, (R)-δ-decanolactone, (S)-γ-dodecanolactone, (R)-γ-dodecanolactone, (S)-8-dodecanolactone, (R)-δ-dodecanolactone, etc.

### 4. Enzymatically Produced Lactones as Building Blocks for Flavors Fragrances and Other Uses

Enzymatically produced lactones such as the enzymatically produced lactones disclosed herein have applications as e.g., fragrances, flavors, nutritional supplements, fuel and etc.

Thus, in some embodiments, enzymatically produced lactones disclosed herein are used alone or in combination with other molecules to provide fragrances and/or flavors for the production of perfume, food, drink, toiletries, etc, nutritional supplements, industrial chemicals, etc.

### III. Preparation of Enzymatically Produced Lactones

### 1. Host Cells and Host Cell Cultures

In view of the present disclosure, the person having ordinary skill in the art will appreciate that any of the embodiments contemplated herein may be practiced with any suitable bacterial host cell that can be genetically modified via the introduction of one or more nucleic acid sequences that code for the appropriate fatty acid biosynthetic enzymes. Accordingly, the recombinant microorganisms disclosed herein function as host cells and comprise one or more polynucleotide sequences that include an open reading frame that encode one or more fatty acid biosynthetic enzymes together with operably-linked regulatory sequences that facilitate heterologous expression of a ybgC protein in the host cell.

Examples of bacteria that provide suitable host cells, include but are not limited to cells from the Class gamma-proteobacteria, cells from the genus Escherichia, Bacillus, Lactobacillus, Zymomonas, Pseudomonas, Marinobacter, or Streptomyces.

In some exemplary embodiments, the host cell is an E. coli cell. In some exemplary embodiments, the E. coli cell is a strain B, a strain C, a strain K, or a strain W E. coli cell.

### 2. Expression of Heterologous Enzymatic Activities in Microorganisms

The expression of enzymatic activities in microorganisms and microbial cells for the production of fatty acid derivative molecules is know in the art and is taught e.g., in the following U.S. Pat. Nos. 9,133,406; 9,340,801; 9,200,299; 9,068,201; 8,999,686; 8,658,404; 8,597,922; 8,535,916; 8,530,221; 8,372,610; 8,323,924; 8,313,934; 8,283,143; 8,268,599; 8,183,028; 8,110,670; 8,110,093; and 8,097,439.

Therefore, in exemplary embodiments, the host cells or host microorganisms that heterologously express a ybgC protein also comprise heterologous enzyme activities that make up pathways for the biosynthetic production of fatty acid derivatives.

Typically recombinant bacteria for the enzymatic production of lactones under physiological conditions heterologously express an acyl-CoA synthetase (FadD) (E.C. 6.2.1.3) activity, in addition to heterologously expressed ybgC protein.

In some embodiments, recombinant bacteria for the enzymatic production of lactones under physiological conditions heterologously express a thioesterase e.g., a thioesterase having activity described by EC 3.1.2 . . . . For example a thioesterase as disclosed in Table 3 (supra).

Typically, the enzymatically produced lactones are recovered from the culture medium and/or are isolated from the host cells. In one exemplary embodiment, the enzymatically produced lactones are recovered from the culture medium (extracellular). In another exemplary embodiment, the enzymatically produced lactones are isolated from the host cells (intracellular). In another exemplary embodiment, the enzymatically produced lactones are recovered from the culture medium and isolated from the host cells.

An enzymatically produced lactone composition produced by a host cell can be analyzed using methods known in the art, for example, Gas-Chromatography with Flame Ionization Detection (GC-FID) in order to determine the distribution of enzymatically produced lactones as well as chain lengths and degree of saturation of the components of the enzymatically produced lactone composition. Similarly, other compounds can be analyzed through methods well known in the art.

### 3. Genetic Alterations for Fine Tuning Recombinant Host Cells

In some exemplary embodiments, host cells comprise optional genetic manipulations and alterations can be used to enhance or otherwise fine tune the production of enzymatically produced lactones molecules. As will be appreciated by a person having ordinary skill in the art, optional genetic manipulations can be used interchangeably from one host cell to another, depending on what other heterologous enzymes and what native enzymatic pathways are present in the host cell. Some optional genetic manipulations are discussed below.

### FadE

FadE (Acyl-CoA dehydrogenase) catalyzes the first step the first step in fatty acid utilization/degradation (β-oxidation cycle) which is the oxidation of acyl-CoA to 2-enoyl-CoA (see e.g., Campbell, J. W. and Cronan, J. E. Jr (2002) J. Bacteriol. 184 (13): 3759-3764, Lennen, R. M. and Pfleger, B. F (2012) Trends Biotechnol. 30 (12): 659-667). Since fadE initiates the β-oxidation cycle, when E. coli lacks FadE, it cannot grow on fatty acids as a carbon source (see e.g., Campbell, J. W. and Cronan supra). The same effect can be achieved by attenuating other enzymes from the β-oxidation cycle, e.g. FadA, which is a 3-ketoacyl-CoA thiolase, or FadB, which is a dual 3-hydroxyacyl-CoA-dehydrogenase/dehydratase.

However, when E. coli is grown on a carbon source other than fatty acids e.g., grown on sugar, acetate, etc., fadE attenuation is optional because under such conditions fadE expression is repressed by FadR. Therefore, when cells are grown on a simple carbon source such as e.g., glucose, the fadE gene product is already attenuated. Accordingly, when grown on a carbon source other than fatty acids, a fadE mutation/deletion is optional.

Overexpression of Non-Native and/or Native and/or Variants of Genes Involved in the Synthesis of Acyl-ACP

In some embodiments, the fatty acid biosynthetic pathway in the production host uses the precursors acetyl-CoA and malonyl-CoA. E. coli or other host organisms engineered to overproduce these components can serve as the starting point for subsequent genetic engineering steps to provide the specific output product (such as, fatty acids, fatty esters, hydrocarbons, fatty alcohols). Several different modifications can be made, either in combination or individually, to the host strain to obtain increased acetyl-CoA/malonyl-CoA/fatty acid and fatty acid derivative production see e.g., US Patent Application Publication 2010/0199548.

Other exemplary modifications of a host cell include e.g., overexpression of non-native and/or native and/or variants of genes involved in the synthesis of acyl-ACP. In general, by increasing acyl-ACP synthesis increases the amount of acyl-ACP, which is the substrate of thioesterases, estersynthases and acyl-ACP reductases. Exemplary enzymes that increase acyl-ACP production include e.g., enzymes that make up the “fatty acid synthase” (FAS). As is known in the art (see e.g., US 2010/0199548) FAS enzymes are a group of enzymes that catalyze the initiation and elongation of acyl chains. The acyl carrier protein (ACP) along with the enzymes in the FAS pathway control the length, degree of saturation, and branching of the fatty acids produced. Enzymes that comprise FAS include e.g., AccABCD, FabD, FabH, FabG, FabA, FabZ, FabI, FabK, FabL, FabM, FabQ, FabV, FabX, FabB, and FabF. Depending upon the desired product one or more of these genes can be attenuated or over-expressed.

Therefore, in exemplary embodiments a host strain may overexpress of one or more of the FAS genes. Exemplary FAS genes that may be overexpressed include e.g., fadR from Escherichia coli (NP_415705.1) fabA from Salmonella typhimurium (NP_460041), fabD from Salmonella typhimurium (NP_460164), fabG from Salmonella typhimurium (NP_460165), fabH from Salmonella typhimurium (NP_460163), fabV from Vibrio cholera (YP_001217283), and fabF from Clostridium acetobutylicum (NP_350156). In some exemplary embodiments, the overexpression of one or more of these genes, which code for enzymes and regulators in fatty acid biosynthesis, serves to further increase the titer of fatty-acid derivative compounds under particular culture conditions. In some exemplary embodiments, the wild-type E. coli strains MG1655 or W3110 (see e.g., Blattner, et al. (1997) 277 (5331): 1453-1462; Jensen, K. F. (1993) J. Bact., 175 (11): 3401-3407) are used as host strains.

### 4. Methods of Making Recombinant Host Cells and Cultures

Any method known in the art can be used to engineer host cells to produce fatty acid derivatives and/or fatty acid derivative compositions or other compounds. Methods for engineering host cells are well known in the art and are readily appreciated and accessible to the skilled practitioner. See e.g., Sambrook et al. (supra); Current Protocols in Molecular Biology (supra).

Generally, a polynucleotide (or gene) sequence is provided to the host cell by way of a recombinant vector that comprises a promoter operably linked to the fatty acid biosynthetic polynucleotide sequence of interest. Once a polynucleotide sequence(s) encoding fatty acid biosynthetic pathway polypeptide has been prepared and isolated, various methods may be used to construct expression cassettes, vectors and other DNA constructs. The skilled artisan is well aware of the genetic elements that must be present on an expression construct/vector in order to successfully transform, select and propagate the expression construct in host cells. Techniques for manipulation of nucleic acids such as subcloning nucleic acid sequences into expression vectors, labeling probes, DNA hybridization, and the like are described generally in e.g., Sambrook, et al., supra; Current Protocols in Molecular Biology, supra.

A number of recombinant vectors are available to those of skill in the art for use in the stable transformation/transfection of bacteria and other microorganisms (see e.g., Sambrook, et al., supra). Appropriate vectors are readily chosen by one of skill in the art.

Once an appropriate vector is identified and constructed, the appropriate transformation technique is readily chosen by the skilled practitioner. Exemplary transformation/transfection methods available to those skilled in the art include e.g., electroporation, calcium chloride transformation and etc., such methods being well known to the skilled artisan (see e.g., Sambrook, supra). In exemplary embodiments, polynucleotide sequences, comprising open reading frames encoding proteins and operably-linked regulatory sequences can be integrated into a chromosome of the recombinant host cells, incorporated in one or more plasmid expression system resident in the recombinant host cells, or both.

As will be appreciated by those skilled in the art, the design of the expression vector can depend on such factors as e.g., the choice of the host cell to be transformed, the level of expression of polypeptide desired, etc.

### IV. Culture and Fermentation of Recombinant Host Cells

As used herein, fermentation broadly refers to the conversion of organic materials into target substances by recombinant host cells. For example, this includes the conversion of a carbon source by recombinant host cells into enzymatically produced lactones as disclosed herein by propagating a culture of the recombinant host cells in a media comprising a carbon source. Conditions permissive for the production of target substances such as e.g., enzymatically produced lactones as disclosed herein, are any conditions that allow a host cell to produce a desired product, such as an enzymatically produced lactone composition. Suitable conditions include, for example, typical fermentation conditions see e.g., Principles of Fermentation Technology, 3rd Edition (2016) supra, Fermentation Microbiology and Biotechnology, 2nd Edition, (2007) supra.

Fermentation conditions can include many parameters, well known in the art, including but not limited to temperature ranges, pH levels, levels of aeration, feed rates and media composition. Each of these conditions, individually and in combination, allows the host cell to grow. Fermentation can be aerobic, anaerobic, or variations thereof (such as micro-aerobic). Exemplary culture media include broths (liquid) or gels (solid). Generally, the medium includes a carbon source (e.g., a simple carbon source derived from a renewable feedstock) that can be metabolized by a host cell directly. In addition, enzymes can be used in the medium to facilitate the mobilization (e.g., the depolymerization of starch or cellulose to fermentable sugars) and subsequent metabolism of the carbon source to enzymatically produce lactones.

For small scale production, the host cells engineered to enzymatically produced lactone compositions are typically grown in batches of, for example, about 100 μL, 200 μL, 300 μL, 400 μL, 500 μL, 1 mL, 5 mL, 10 mL, 15 mL, 25 mL, 50 mL, 75 mL, 100 mL, 500 mL, 1 L, 2 L, 5 L, or 10 L.

For large scale production, the engineered host cells can be grown in cultures having a volume batches of about 10 L, 100 L, 1000 L, 10,000 L, 100,000 L, 1,000,000 L or larger; fermented; and induced to express any desired polynucleotide sequence.

The non-native monounsaturated fatty acid derivative compositions disclosed herein can be found in the extracellular environment of the recombinant host cell culture and can be readily isolated from the culture medium by methods known in the art. A non-native monounsaturated fatty acid derivative may be secreted by the recombinant host cell, transported into the extracellular environment or passively transferred into the extracellular environment of the recombinant host cell culture.

Exemplary microorganisms suitable for use as production host cells for the production of the enzymatically produced lactones include e.g., bacteria, cyanobacteria, etc. To produce fatty acid derivative compositions production host cells (or equivalently, host cells) are engineered to comprise fatty acid biosynthesis pathways that are modified relative to non-engineered or native host cells e.g., engineered as discussed above and as disclosed e.g., in U.S. Patent Application Publication 2015/0064782. Production hosts engineered to comprise modified fatty acid biosynthesis pathways are able to efficiently convert glucose or other renewable feedstocks into fatty acid derivatives. Protocols and procedures for high density fermentations for the production of various compounds have been established (see, e.g., U.S. Pat. Nos. 8,372,610; 8,323,924; 8,313,934; 8,283,143; 8,268,599; 8,183,028; 8,110,670; 8,110,093; and 8,097,439).

In some exemplary embodiments, a production host cell is cultured in a culture medium (e.g., fermentation medium) comprising an initial concentration of a carbon source (e.g., a simple carbon source) of about 20 g/L to about 900 g/L. In other embodiments, the culture medium comprises an initial concentration of a carbon source of about 2 g/L to about 10 g/L; of about 10 g/L to about 20 g/L; of about 20 g/L to about 30 g/L; of about 30 g/L to about 40 g/L; or of about 40 g/L to about 50 g/L. In some embodiments, the level of available carbon source in the culture medium can be monitored during the fermentation proceeding. In some embodiments, the method further includes adding a supplemental carbon source to the culture medium when the level of the initial carbon source in the medium is less than about 0.5 g/L.

In some exemplary embodiments, a supplemental carbon source is added to the culture medium when the level of the carbon source in the medium is less than about 0.4 g/L, less than about 0.3 g/L, less than about 0.2 g/L, or less than about 0.1 g/L. In some embodiments, the supplemental carbon source is added to maintain a carbon source level of about 1 g/L to about 25 g/L. In some embodiments, the supplemental carbon source is added to maintain a carbon source level of about 2 g/L or more (e.g., about 2 g/L or more, about 3 g/L or more, about 4 g/L or more). In certain embodiments, the supplemental carbon source is added to maintain a carbon source level of about 5 g/L or less (e.g., about 5 g/L or less, about 4 g/L or less, about 3 g/L or less). In some embodiments, the supplemental carbon source is added to maintain a carbon source level of about 2 g/L to about 5 g/L, of about 5 g/L to about 10 g/L, or of about 10 g/L to about 25 g/L.

In one exemplary embodiment the carbon source for the fermentation is derived from a renewable feedstock. In some embodiments, the carbon source is glucose. In other embodiments, the carbon source is glycerol. Other possible carbon sources include, but are not limited to, fructose, mannose, galactose, xylose, arabinose, starch, cellulose, pectin, xylan, sucrose, maltose, cellobiose, and turanose; cellulosic material and variants such as hemicelluloses, methyl cellulose and sodium carboxymethyl cellulose; saturated or unsaturated fatty acids, succinate, lactate, and acetate; alcohols, such as ethanol, methanol, and glycerol, or mixtures thereof. In one embodiment, the carbon source is derived from corn, sugar cane, sorghum, beet, switch grass, ensilage, straw, lumber, pulp, sewage, garbage, cellulosic urban waste, flu-gas, syn-gas, or carbon dioxide. The simple carbon source can also be a product of photosynthesis, such as glucose or sucrose. In one embodiment, the carbon source is derived from a waste product such as glycerol, flu-gas, or syn-gas; or from the reformation of organic materials such as biomass; or from natural gas or from methane, or from the reformation of these materials to syn-gas; or from carbon dioxide that is fixed photosynthetically, for example enzymatically produced lactones may be produced by recombinant cyanobacteria growing photosynthetically and using CO₂ as carbon source. In some exemplary embodiments, the carbon source is derived from biomass. An exemplary source of biomass is plant matter or vegetation, such as corn, sugar cane, or switchgrass. Another exemplary source of biomass is metabolic waste products, such as animal matter (e.g., cow manure). Further exemplary sources of biomass include algae and other marine plants. Biomass also includes waste products from industry, agriculture, forestry, and households, including, but not limited to, fermentation waste, ensilage, straw, lumber, sewage, garbage, cellulosic urban waste, municipal solid waste, and food leftovers.

In some exemplary embodiments, enzymatically produced lactones is produced at a concentration of about 0.5 g/L to about 40 g/L. In some embodiments, a fatty acid derivative is produced at a concentration of about 1 g/L or more (e.g., about 1 g/L or more, about 10 g/L or more, about 20 g/L or more, about 50 g/L or more, about 100 g/L or more). In some embodiments, a fatty acid derivative is produced at a concentration of about 1 g/L to about 170 g/L, of about 1 g/L to about 10 g/L, of about 40 g/L to about 170 g/L, of about 100 g/L to about 170 g/L, of about 10 g/L to about 100 g/L, of about 1 g/L to about 40 g/L, of about 40 g/L to about 100 g/L, or of about 1 g/L to about 100 g/L.

In other exemplary embodiments, enzymatically produced lactones are produced at a titer of about 25 mg/L, about 50 mg/L, about 75 mg/L, about 100 mg/L, about 125 mg/L, about 150 mg/L, about 175 mg/L, about 200 mg/L, about 225 mg/L, about 250 mg/L, about 275 mg/L, about 300 mg/L, about 325 mg/L, about 350 mg/L, about 375 mg/L, about 400 mg/L, about 425 mg/L, about 450 mg/L, about 475 mg/L, about 500 mg/L, about 525 mg/L, about 550 mg/L, about 575 mg/L, about 600 mg/L, about 625 mg/L, about 650 mg/L, about 675 mg/L, about 700 mg/L, about 725 mg/L, about 750 mg/L, about 775 mg/L, about 800 mg/L, about 825 mg/L, about 850 mg/L, about 875 mg/L, about 900 mg/L, about 925 mg/L, about 950 mg/L, about 975 mg/L, about 1000 mg/L, about 1050 mg/L, about 1075 mg/L, about 1100 mg/L, about 1125 mg/L, about 1150 mg/L, about 1175 mg/L, about 1200 mg/L, about 1225 mg/L, about 1250 mg/L, about 1275 mg/L, about 1300 mg/L, about 1325 mg/L, about 1350 mg/L, about 1375 mg/L, about 1400 mg/L, about 1425 mg/L, about 1450 mg/L, about 1475 mg/L, about 1500 mg/L, about 1525 mg/L, about 1550 mg/L, about 1575 mg/L, about 1600 mg/L, about 1625 mg/L, about 1650 mg/L, about 1675 mg/L, about 1700 mg/L, about 1725 mg/L, about 1750 mg/L, about 1775 mg/L, about 1800 mg/L, about 1825 mg/L, about 1850 mg/L, about 1875 mg/L, about 1900 mg/L, about 1925 mg/L, about 1950 mg/L, about 1975 mg/L, about 2000 mg/L (2 g/L), 3 g/L, 5 g/L, 10 g/L, 20 g/L, 30 g/L, 40 g/L, 50 g/L, 60 g/L, 70 g/L, 80 g/L, 90 g/L, 100 g/L or a range bounded by any two of the foregoing values. In other embodiments, a fatty acid derivative or other compound is produced at a titer of more than 100 g/L, more than 200 g/L, or more than 300 g/L. In exemplary embodiments, the titer of fatty acid derivative or other compound produced by a recombinant host cell according to the methods disclosed herein is from 5 g/L to 200 g/L, 10 g/L to 150 g/L, 20 g/L to 120 g/L and 30 g/L to 100 g/L. The titer may refer to a particular fatty acid derivative or a combination of fatty acid derivatives or another compound or a combination of other compounds produced by a given recombinant host cell culture. In exemplary embodiments, the expression of ChFatB2 thioesterase variant in a recombinant host cell such as E. coli results in the production of a higher titer as compared to a recombinant host cell expressing the corresponding wild type polypeptide. In one embodiment, the higher titer ranges from at least about 5 g/L to about 200 g/L.

In other exemplary embodiments, the host cells engineered to produce enzymatically produced lactones according to the methods of the disclosure have a yield of at least 1%, at least 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, or at least about 30% or a range bounded by any two of the foregoing values. In other embodiments, a fatty acid derivative or derivatives or other compound(s) are produced at a yield of more than about 30%, more than about 35%, more than about 40%, more than about 45%, more than about 50%, more than about 55%, more than about 60%, more than about 65%, more than about 70%, more than about 75%, more than about 80%, more than about 85%, more than about 90%, more than 100%, more than 200%, more than 250%, more than 300%, more than 350%, more than 400%, more than 450%, more than 500%, more than 550%, more than 600%, more than 650%, more than 700%, more than 750%, or more. Alternatively, or in addition, the yield is about 30% or less, about 27% or less, about 25% or less, or about 22% or less. In another embodiment, the yield is about 50% or less, about 45% or less, or about 35% or less. In another embodiment, the yield is about 95% or less, or 90% or less, or 85% or less, or 80% or less, or 75% or less, or 70% or less, or 65% or less, or 60% or less, or 55% or less, or 50% or less. Thus, the yield can be bounded by any two of the above endpoints. For example, the yield of an enzymatically produced lactone e.g., an γ-,δ-, or ε-lactone, produced by the recombinant host cell according to the methods disclosed herein can be about 5% to about 15%, about 10% to about 25%, about 10% to about 22%, about 15% to about 27%, about 18% to about 22%, about 20% to about 28%, about 20% to about 30%, about 30% to about 40%, about 40% to about 50%, about 50% to about 60%, about 60% to about 70%, about 70% to about 80%, about 80% to about 90%, about 90% to about 100%, about 100% to about 200%, about 200% to about 300%, about 300% to about 400%, about 400% to about 500%, about 500% to about 600%, about 600% to about 700%, or about 700% to about 800%. The yield may refer to a particular enzymatically produced lactone or a combination of enzymatically produced lactones. In one embodiment, the higher yield ranges from about 10% to about 800% of theoretical yield. In addition, the yield will also be dependent on the feedstock used.

In some exemplary embodiments, the productivity of the host cells engineered to produce an enzymatically produced lactone according to the methods of the disclosure is at least 100 mg/L/hour, at least 200 mg/L/hour, at least 300 mg/L/hour, at least 400 mg/L/hour, at least 500 mg/L/hour, at least 600 mg/L/hour, at least 700 mg/L/hour, at least 800 mg/L/hour, at least 900 mg/L/hour, at least 1000 mg/L/hour, at least 1100 mg/L/hour, at least 1200 mg/L/hour, at least 1300 mg/L/hour, at least 1400 mg/L/hour, at least 1500 mg/L/hour, at least 1600 mg/L/hour, at least 1700 mg/L/hour, at least 1800 mg/L/hour, at least 1900 mg/L/hour, at least 2000 mg/L/hour, at least 2100 mg/L/hour, at least 2200 mg/L/hour, at least 2300 mg/L/hour, at least 2400 mg/L/hour, 2500 mg/L/hour, or as high as 10 g/L/hour (dependent upon cell mass). The productivity may refer to a particular enzymatically produced lactones e.g., a γ-lactone, or a combination of enzymatically produced lactones or other compound(s) produced by a given host cell culture. For example, the heterologous expression of a YbgC protein and an acyl-CoA synthetase in a recombinant host cell such as E. coli results in increased productivity of the enzymatically produced lactones as compared to a recombinant host cell that does not heterologously express (overexpress a native E. coli YbgC or heterologous YbgC from and organism other than E. coli) a YbgC protein and an acyl-CoA synthetase. In exemplary embodiments, higher productivity ranges from about 0.3 g/L/h to about 3 g/L/h to about 10 g/L/h to about 100 g/L/h to about a 1000 g/L/h.

As disclosed supra, in some exemplary embodiments, the host cell used in the fermentation procedures discussed herein (supra) is a mammalian cell, plant cell, insect cell, yeast cell, fungus cell, filamentous fungi cell, an algal cell, a cyanobacterial cell, and bacterial cell.

### V. Isolation

Bioproducts e.g., compositions comprising enzymatically produced lactones as disclosed herein which are produced utilizing recombinant host cells as discussed above are typically isolated from the fermentation broth by methods known in the art. In an exemplary embodiment the compositions comprising the enzymatically produced lactones as disclosed herein which are produced utilizing recombinant host cells are discussed above are isolated from the fermentation broth by gravity settling, centrifugation, or decantation.

### VI. Compositions and Formulations of Enzymatically Produced Lactones

Bioproducts e.g., compositions comprising enzymatically produced lactones produced utilizing engineered bacteria as discussed herein, are produced from renewable sources (e.g., from a simple carbon source derived from renewable feedstocks) and, as such, are new compositions of matter. These new bioproducts can be distinguished from organic compounds derived from petrochemical carbon on the basis of dual carbon-isotopic fingerprinting or ¹⁴C dating. Additionally, the specific source of biosourced carbon (e.g., glucose vs. glycerol) can be determined by dual carbon-isotopic fingerprinting by methods known in the art (see, e.g., U.S. Pat. No. 7,169,588, WO 2016/011430 A1, etc.).

The following examples are offered to illustrate, but not to limit the invention.

### EXAMPLES

### Example 1

The following Example illustrates materials and methods for Examples 2-9 disclosed herein below.

### Methods

### Small Scale Fermentation Protocol:

40 μL LB culture (from an LB culture growing in a 96 well plate) was used to inoculate 360 μL LB media, which was then incubated for approximately 4 hours at 32° C. shaking. 80 μL of the LB seed was used to inoculate 320 μL Nlim media (Table 5). After growing at 32° C. for 2 hours, the cultures were induced with IPTG (final concentration 1 mM). The cultures were then incubated at 32° C. with shaking for 20 hours if not noted otherwise, after which they were extracted following the standard extraction protocol detailed below.

TABLE 5
N-lim Media Formulation
N-lim Media Formulation

| | | |
|---|---|---|
| 1 | x | 5x Salt Soln. with NH4Cl |
| 1 | x | 1000x Trace Vitamins |
| 1 | mg/L | 10 mg/mL Thiamine |
| 1 | mM | 1M MgSO4 |
| 0.1 | mM | 1M CaCl2 |
| 40 | g/L | 500 g/L glucose |
| 1 | x | 1000x Trace minerals |
| 10 | mg/L | 10 g/L Fe Citrate |
| 100 | μg/mL | 100 mg/ml spectinomycin |
| 100 | mM | 2M BisTris (pH 7.0) |
| 0.5 | mM | Aminolevulinic acid |

### Fatty Acid Species Standard Extraction and Analytical Protocol

To each well to be extracted, 80 μL of 1M HCl, followed by 400 μL of butyl acetate containing 500 mg/L 1-undecanol or 500 mg/L undecanoic acid as internal standard (IS) was added as internal standard (IS) was added. The 96 well plates were then heat-sealed using a plate sealer (ALPS-300 heater; Abgene, ThermoScientific, Rockford, IL), and shaken for 15 minutes at 2000 rpm using MIXMATE mixer (Eppendorf, Hamburg, Germany). After shaking, the plates were centrifuged for 10 minutes at 4500 rpm at room temperature (Allegra X-15R, rotor SX4750A, Beckman Coulter, Brea, CA) to separate the aqueous and organic layers. 50 μL of the organic layer was transferred to a 96 well plate (polypropylene, Corning, Amsterdam, The Netherlands) and derivatized with 50 μL of trimethylsiloxy/N,O-Bis (trimethylsilyl)trifluoroacetamide (TMS/BSTFA). The plate was subsequently heat sealed and stored at −20° C. until evaluated by either Gas Chromatography with Flame Ionization Detection (GC-FID) or Gas Chromatography-Mass Spectrometry (GC-MS).

The GC-MS parameters used to generate chromatograms and mass spectra for compounds identification were as follows: 1 μl sample was injected into analytical Column: DB-1HT, 15 m×250 μm×0.1 μm, available from Agilent with cat #J&W 122-1111E, Oven temperature: initial at 50° C., hold for 5 minutes, increase to 300° C. at 25° C./min, and hold for 5.24 minutes for a total run time of 24 minutes. Column flow: 1.2 mL/min, Inlet temperature: 300° C., Split ratio: 20:1, Software: ChemStation E.02.01.1177. MS parameters: Transfer line temperature: 300° C., MS source: 230° C., MS Quad: 150° C. Auto sampler: Combi PAL (CTC analytics) distributed by LEAP Technologies. The GC-FID parameters used to quantify each compound were carried out as follows: 1 μL of sample was injected onto an analytical column (UFC Rtx-1, 5 M×0.1 mm×0.1 μM) in a Thermo Fisher UltraFast TRACE GC (Thermo Fisher Scientific, West Palm Beach, FL). Oven temperature: initial at 100° C., hold for 0.2 minutes, increase to 320° C. at 100° C./min, and hold for 0.5 minutes for a total run time of 2.5 minutes using column flow of 0.5 ml/min, Inlet temperature: 300° C. and flame ionization detector temperature: 300° C.

The protocol detailed above represents standard conditions, a person having ordinary skill in the art appreciates that the protocol may be modified to optimize the analytical results.

TABLE 6
Strains used for bioconversion of hydroxy fatty acids to lactones

| Strain | |
|---|---|
| Name | Description |
| DS08-004 | MG1655 ΔfadD |
| sKM.564 | MG1655 ΔfadD ΔybgC |
| VA1052 | MG1655 ΔfadD::Ptrc-fadD3_Pput |
| sKM.565 | MG1655 ΔfadD::Ptrc-fadD3_Pput, ΔybgC |
| sKM.589 | DS08-004 with empty pCL plasmid |
| sKM.590 | DS08-004 with pKM.122 (pCL-Ptrc-ybgC) |
| sKM.591 | sKM.564 with empty pCL plasmid |
| sKM.528 | VA1052 with empty pCL plasmid |
| sKM.593 | sKM.565 with empty pCL plasmid |
| sKM.529 | VA1052 with plasmid pKM.122 (pCL-Ptrc-ybgC) |
| sKM.594 | sKM.565 with plasmid pKM.122 (pCL-Ptrc-ybgC) |

### Example 2

The following Example illustrates conversion of 4-hydroxydecanoic acid to γ-decalactone by E. coli strains expressing acyl CoA synthase and ybgC.

This Example shows the conversion of 4-hydroxydecanoic acid to γ-decalactone by recombinant E. coli MG1655 derivative strains expressing acyl-CoA synthetase and ybgC. Several base strains were created by deleting from the chromosome either the endogenous acyl-CoA synthase gene, fadD, or the endogenous ybgC gene, or by deleting both genes (see Table 6). Additionally in selected strains, the fadD3 gene from Pseudomonas putida was cloned under the control of the IPTG-inducible Ptrc promoter and integrated into the fadD locus for heterologous expression from the E. coli chromosome (see Table 6). The YbgC gene was amplified from genomic DNA and cloned into a pCL-derivative vector (SC101 replicon, spectinomycin resistance marker) such that it was under the control of the IPTG-inducible Ptrc. The plasmid was named pKM. 122.

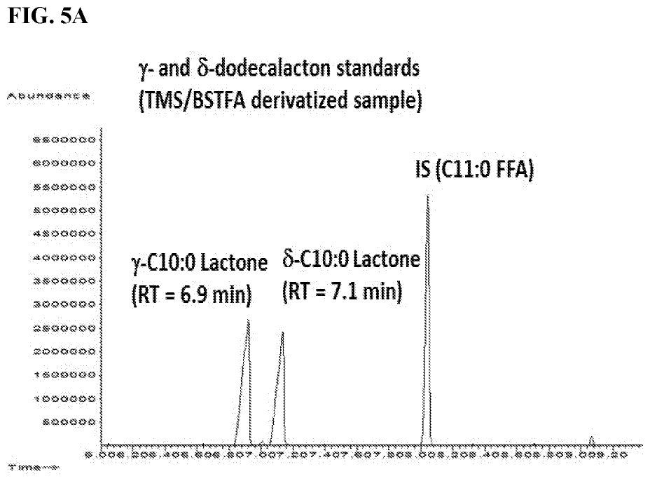

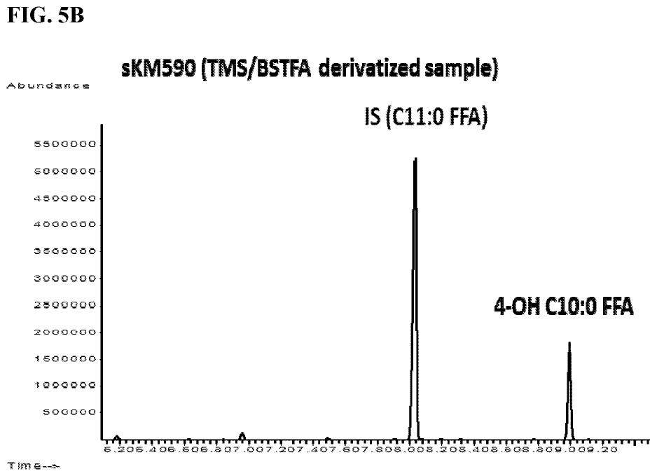

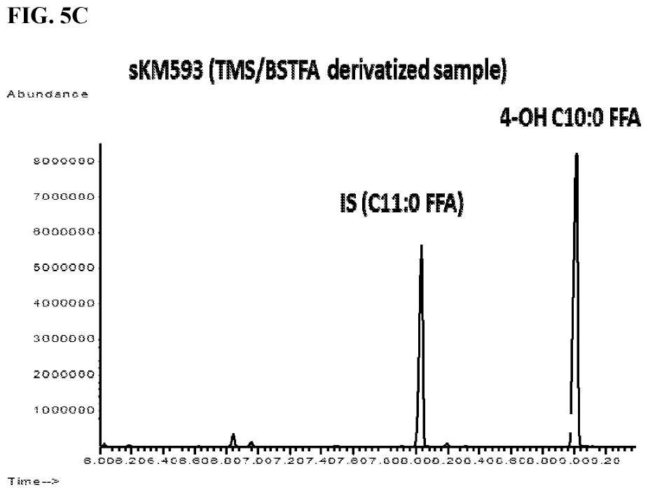

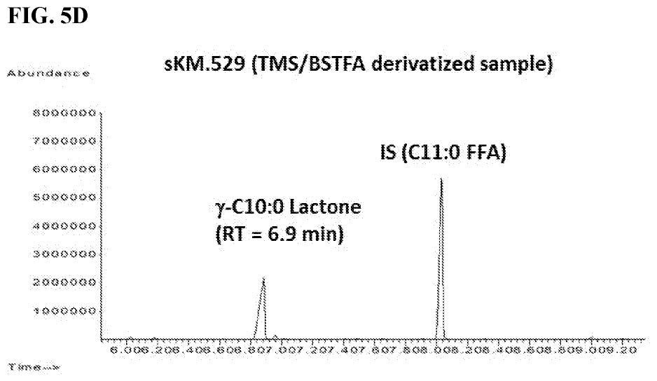

Plasmid pKM.122 or the empty pCL-control plasmid were transformed into the base strains and the resulting strains (see Table 6) were then grown as disclosed in Example 1 and 4-hydroxydecanoic acid was added to culture media as disclosed in Example 1. After 20 h, the cultures were extracted as disclosed in Example 1. The chromatographs of the extracts from three strains compared to authentic γ- and δ-decalactone are shown in FIG. 5. Strains with deletion of the endogenous acyl-CoA-dehydrogenase and ybgC genes, sKM.589 and sKM.591, showed only trace amounts of γ-decalactone. Similarly, the strain with the ybgC gene deleted and FadD3 from P. putida overexpressed (sKM.593) showed only trace amounts of γ-decalactone (see Table 7). These results demonstrate that 4-hydroxydecanoic acid or 4-hydroxydecanoyl-CoA do not spontaneously lactonize to form γ-decalactone without acidification of the culture broth.

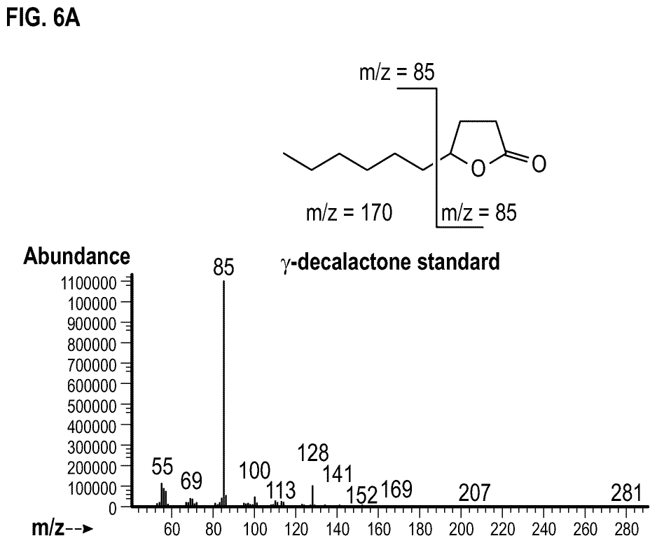

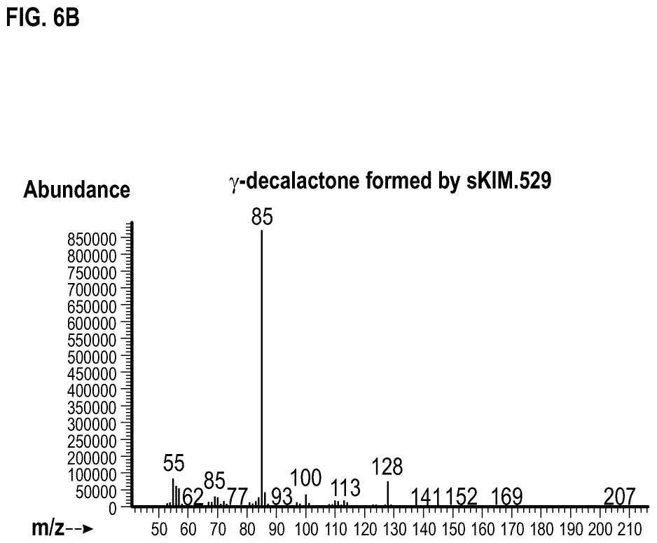

In contrast, the strain with the intact chromosomal copy of the ybgC gene and FadD3 from P. putida overexpressed, sKM.528, showed 82% conversion of 4-hydroxydecanoic acid to γ-decalactone, and the two strains with FadD3 from P. putida and YbgC overexpressed, sKM.529 and sKM.594, showed complete (100%) and almost complete (96%) conversion of 4-hydroxydecanoic acid to γ-decalactone, respectively (see Table 7). The new peak at RT=6.9 min was identified as γ-decalactone by its retention time (FIG. 5) and fragmentation pattern (see FIG. 6), which matched the authentic standard.

These results demonstrated that the combined expression of YbgC and FadD3 from P. putida were sufficient to convert 4-hydroxydecanoic acid to γ-decalactone in-vivo, i.e. under physiological conditions without the need to acidify the culture broth.

TABLE 7
Bioconversion of 4-hydroxydecanoic acid to
γ-decalactone in recombinant E. coli.

| | Deleted | Overexpressed | Conversion |
|---|---|---|---|
| Strain | gene | genes | (%) |
| MG1655 | — | — | 0 |
| sKM.589 | fadD | — | 1 |
| sKM.590 | fadD | ybgC | 3 |
| sKM.591 | fadD, ybgC | — | 2 |
| sKM.528 | — | fadD3 | 82 |
| sKM.529 | — | fadD3, ybgC | 100 |
| sKM.593 | ybgC | fadD3 | 1 |
| sKM.594 | ybgC | fadD3, ybgC | 96 |

### Example 3

The following Example illustrates conversion of 5-hydroxydecanoic acid to 8-decalactone by E. coli strains expressing acyl CoA synthetase and ybgC.

This Example shows the conversion of exogenously added 5-hydroxydecanoic acid to δ-decalactone by recombinant E. coli strains expressing acyl-CoA synthetase and ybgC. The same strains as in Example 2 were used for this example.

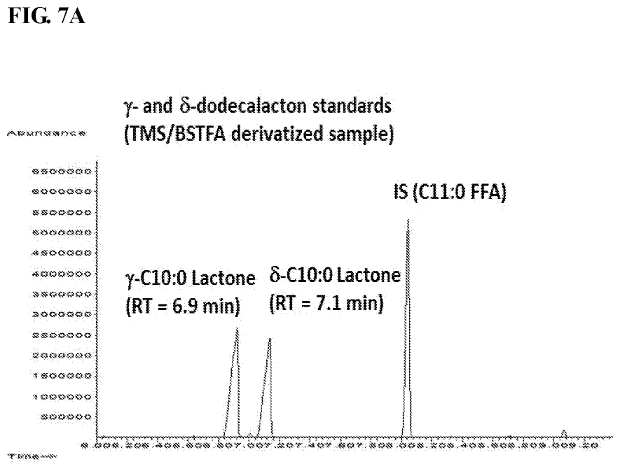

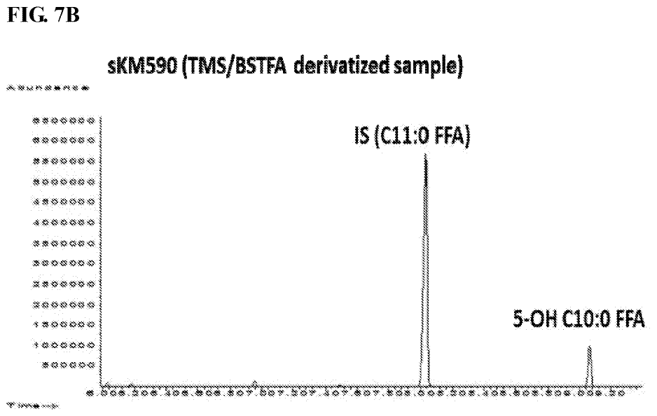

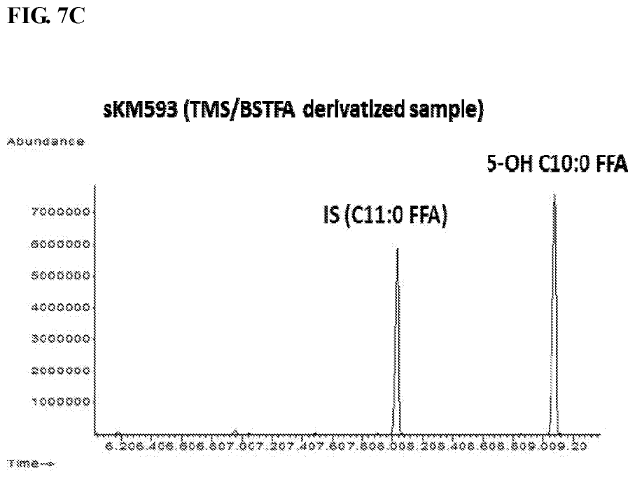

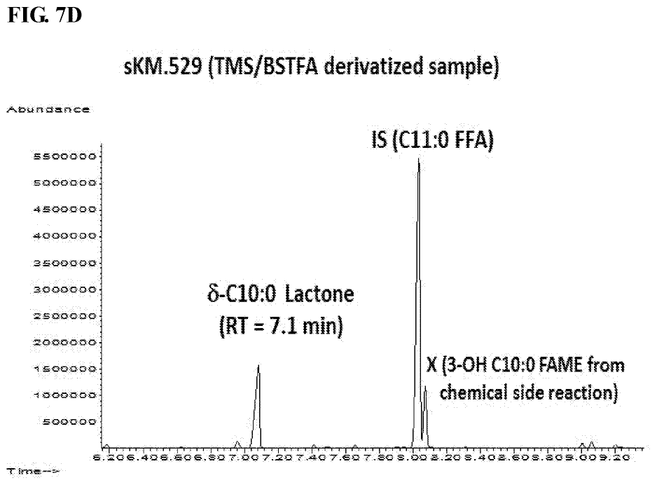

The strains (see Table 6) were grown as disclosed in Example 1 and 5-hydroxydecanoic acid was added to culture media as disclosed in Example 1. After 20 h, the cultures were extracted as disclosed in Example 1. The chromatographs of the extracts from three strains compared to authentic γ- and δ-decalactone are shown in FIG. 7.

Strains with deletion of the endogenous acyl-CoA-dehydrogenase and ybgC genes, sKM.589 and sKM.591, showed no detectable amounts of δ-decalactone. Similarly, the strain with the ybgC gene deleted and FadD3 from P. putida overexpressed (sKM.593) showed no detectable amounts of δ-decalactone (see Table 8). These results demonstrate that 5-hydroxydecanoic acid or 5-hydroxydecanoyl-CoA do not spontaneously lactonize to form δ-decalactone without acidification of the culture broth.

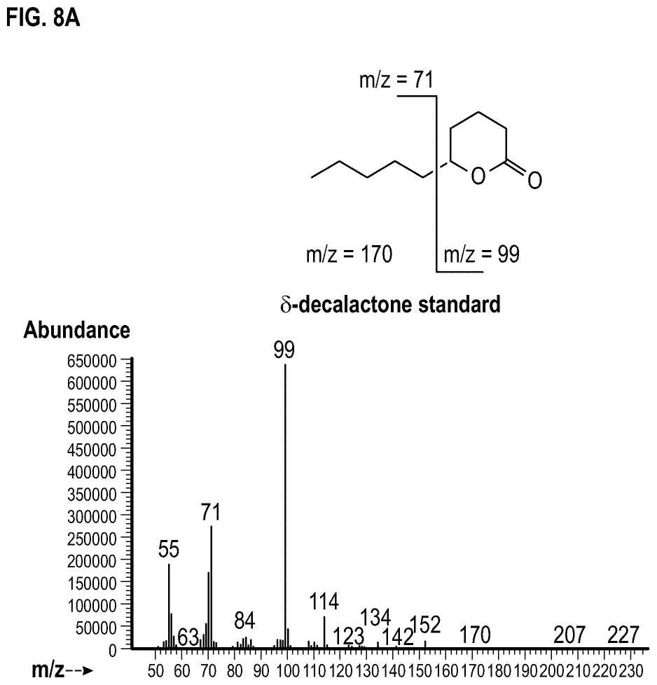

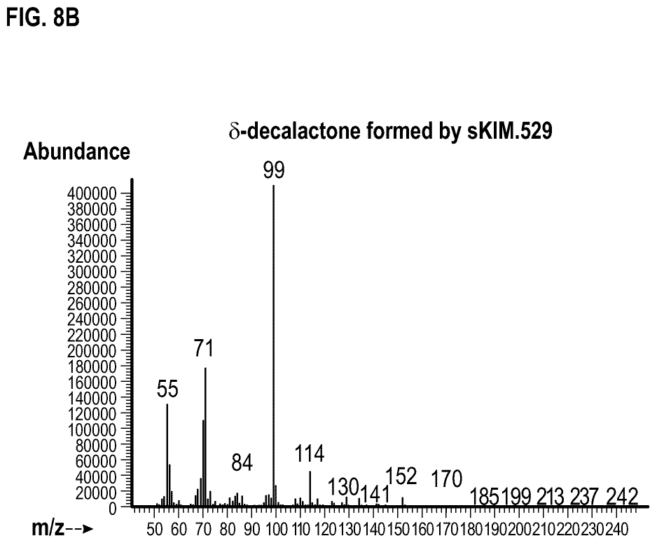

In contrast, the strain with the intact chromosomal copy of the ybgC gene and FadD3 from P. putida overexpressed, sKM.528, showed 10% conversion of 5-hydroxydecanoic acid to δ-decalactone, and the two strains with FadD3 from P. putida and YbgC overexpressed, sKM.529 and sKM.594, showed almost complete (91 and 96%) conversion of 5-hydroxydecanoic acid to δ-decalactone (see Table 8). The new peak at RT=7.1 min was identified as δ-decalactone by its retention time (FIG. 7) and fragmentation pattern (see FIG. 8), which matched the authentic standard.

These results demonstrated that the combined expression of YbgC and FadD3 from P. putida were sufficient to convert 5-hydroxydecanoic acid to δ-decalactone in-vivo, i.e. under physiological conditions without the need to acidify the culture broth.

TABLE 8
Bioconversion of 5-hydroxydecanoic acid to
δ-decalactone in recombinant E. coli.

| | Deleted | Overexpressed | Conversion |
|---|---|---|---|
| Strain | genes | genes | (%) |
| MG1655 | — | — | 0 |
| sKM.589 | fadD | — | 0 |
| sKM.590 | fadD | ybgC | 0 |
| sKM.591 | fadD, ybgC | — | 0 |
| sKM.528 | — | fadD3 | 10 |
| sKM.529 | — | fadD3, ybgC | 91 |
| sKM.593 | ybgC | fadD3 | 0 |
| sKM.594 | ybgC | fadD3, ybgC | 96 |

### Example 4

The following Example illustrates conversion of 6-hydroxyhexanoic acid to ε-lactone by E. coli strains expressing acyl CoA synthetase and ybgC.

This Example shows the conversion of exogenously added 6-hydroxyhexanoic acid to α-lactone by recombinant E. coli strains expressing acyl-CoA synthetase and ybgC. Strains from Example 2 were used for this Example.

The strains (see Table 6) were grown as disclosed in Example 1 and 6-hydroxyhexanoic acid was added to culture media as disclosed in Example 1. After 20 h, the cultures were extracted as disclosed in Example 1. The chromatographs of the extracts from two strains were compared to authentic δ-caprolactone.

The strain with the ybgC gene deleted and FadD3 from P. putida overexpressed (sKM.593) showed no detectable amounts of 8-caprocalactone (see Table 9). These results demonstrate that 6-hydroxyhexanoic acid or 6-hydroxyhexanoyl-CoA do not spontaneously lactonize to form α-lactone without acidification of the culture broth.

In contrast, the strain with FadD3 from P. putida and YbgC overexpressed, sKM.594, showed 52% conversion of 6-hydroxyhexanoic acid to α-lactone (see Table 9). The new peak was identified as ε-caprolactone by its retention time and fragmentation pattern, which matched the authentic standard (data not shown).

These results demonstrated that the combined expression of YbgC and FadD3 from P. putida were sufficient to convert 6-hydroxyhexanoic acid to ε-caprolactone in-vivo, i.e. under physiological conditions without the need to acidify the culture broth.

TABLE 9
Bioconversion of 6-hydroxyhexanoic acid to
ε-caprolactone in recombinant E. coli.

| | Deleted | Overexpressed | Conversion |
|---|---|---|---|
| Strain | genes | genes | (%) |
| sKM.593 | ybgC | fadD3 | 0 |
| sKM.594 | ybgC | fadD3, ybgC | 52 |

### Example 5

The following Example illustrates conversion of 4-hydroxydecanoic acid to γ-decalactone by E. coli strains expressing acyl CoA synthase and homologs of ybgC.

This Example shows the conversion of 4-hydroxydecanoic acid to γ-decalactone by recombinant E. coli MG1655 derivative strains expressing acyl-CoA synthetase and YbgC homologs from other microorganisms. The YbgC homologs tested in this experiment and their percent identity to YbgC from E. coli are given in Table 2.

Five homologous ybgC genes were synthesized and cloned into a pCL-derivative vector (SC101 replicon, spectinomycin resistance marker) such that they were under the control of the IPTG-inducible Ptrc.

The plasmids were transformed into the base strains sKM.565 (see Table 6) and the resulting strains were then grown as disclosed in Example 1 and 4-hydroxydecanoic acid was added to culture media as disclosed in Example 1. After 20 h, the cultures were extracted as disclosed in Example 1 and the amount of γ-decalactone and the residual amount of 4-hydroxydecanoic acid were determined

As can be seen in Table 10, all five tested YbgC homologs converted 4-hydroxydecanoic acid (via 4-hydroxydecanyl-CoA in the presence of FadD3 from P. putida) to γ-decalactone. For example, the recombinant E. coli strain expressing the YbgC homolog from Plesiomonas shigelloides, which is only 62% identical to YbgC from E. coli, converted 74% of 4-hydroxydecanoic acid to γ-decalactone. Table 2 shows the percent identity and FIG. 9 shows an alignment of these six enzymes of the ybgC protein family.

These results demonstrated that the YbgC family contains enzymes that in the presence of on acyl-CoA synthetase convert 4-hydroxydecanoic acid to γ-decalactone in-vivo, i.e. under physiological conditions without the need to acidify the culture broth.

TABLE 10
Bioconversion of 4-hydroxydecanoic acid to
γ-decalactone in recombinant E. coli.

| overexpressed | | |
|---|---|---|
| Gene | Organism | Conversion % |
| ybgC | Escherichia coli | 87% |
| | (SEQ ID NO: 1) | |
| none | — | 0 |
| ybgC | Citrobacter koseri | 93% |
| | (SEQ ID NO: 2) | |
| ybgC | Enterobacter cloacae(SEQ | 76% |
| | ID NO: 3) | |
| Acyl-CoA | Serratia fonticola | 72% |
| thioesterase | (SEQ ID NO: 4) | |
| Acyl-CoA | Exiguobacterium mexicanum | 36% |
| thioesterase | (SEQ ID NO: 5) | |
| ybgC | Plesiomonas shigelloides | 74% |
| | (SEQ ID NO: 6) | |

### Example 6

The following Example illustrates conversion of 5-hydroxydecanoic acid to 8-decalactone by E. coli strains expressing acyl CoA synthetase and homologs of ybgC.

This Example shows the conversion of 5-hydroxydecanoic acid to δ-decalactone by recombinant E. coli MG1655 derivative strains expressing acyl-CoA synthetase and YbgC homologs from other microorganisms. The YbgC homologs tested in this experiment and their percent identity to YbgC from E. coli are given in Table 2.

Five homologous ybgC genes were synthesized and cloned into a pCL-derivative vector (SC101 replicon, spectinomycin resistance marker) such that they were under the control of the IPTG-inducible Ptrc.

The plasmids were transformed into the base strain sKM.565 (see Table 6) and the resulting strains were then grown as disclosed in Example 1 and 5-hydroxydecanoic acid was added to culture media as disclosed in Example 1. After 20 h, the cultures were extracted as disclosed in Example 1 and the amount of δ-decalactone and the residual amount of 5-hydroxydecanoic acid were determined

As can be seen in Table 11, all five tested YbgC homologs converted 5-hydroxydecanoic acid (via 5-hydroxydecanyl-CoA in the presence of FadD3 from P. putida) to δ-decalactone. For Example, the recombinant E. coli strain expressing the YbgC homolog from Plesiomonas shigelloides, which is only 62% identical to YbgC from E. coli, converted 80% of 5-hydroxydecanoic acid to δ-decalactone. Table 2 shows the percent similarity and FIG. 10 shows an alignment of these six enzymes of the ybgC protein family.

These results demonstrated that the YbgC family contains enzymes that in the presence of on acyl-CoA synthetase convert 5-hydroxydecanoic acid to δ-decalactone in-vivo, i.e. under physiological conditions without the need to acidify the culture broth.

TABLE 11
Bioconversion of 5-hydroxydecanoic acid to
δ-decalactone in recombinant E. coli.

| overexpressed | | |
|---|---|---|
| Gene | Organism | Conversion % |
| ybgC | Escherichia coli | 96% |
| none | — | 2% |
| ybgC | Citrobacter koseri | 85% |
| ybgC | Enterobacter cloacae | 25% |
| Acyl-CoA thioesterase | Serratia fonticola | 34% |
| Acyl-CoA thioesterase | Exiguobacterium mexicanum | 36% |
| ybgC | Plesiomonas shigelloides | 80% |

### Example 7

The following Example illustrates conversion of hydroxyl fatty acid to lactones by E. coli strains expressing acyl CoA synthetase and ybgC (D18A) variant.

This Example shows the conversion of 4-hydroxydecanoic acid, 5-hydroxydecanoic acid and 6-hydroxyhexanoic acid to the respective lactones by recombinant E. coli MG1655 derivative strains expressing acyl-CoA synthetase and a YbgC variant enzyme with aspartate in position 18 mutated to alanine.

Codon 18 of the E. coli ybgC was changed from GAT to GCG using standard molecular biology techniques. The mutated gene, which encodes a mutated ybgC (D18A) protein, was cloned into a pCL-derivative vector (SC101 replicon, spectinomycin resistance marker) such that it was under the control of the IPTG-inducible Ptrc, and the resulting plasmid was transformed into strain sKM.565 (see Table 6)

The resulting strain and the control strain sKM.594 (see Table 6) expressing wildtype YbgC were then grown as disclosed in Example 1 and 4-hydroxydecanoic acid, 5-hydroxydecanoic acid or 6-hydroxyhexanoic acid were added to culture media as disclosed in Example 1. After 20 h, the cultures were extracted as disclosed in Example 1 and the amount of γ- or δ-decalactone or ε-caprolactone and the residual amount of 4- or 5-hydroxydecanoic acid or 6-hydroxyhexanoic acid were determined.

As can be seen in Table 12, the mutated YbgC (D18A) enzyme converted only 30% of 4-hydroxydecanoic acid (via 4-hydroxydecanyl-CoA in the presence of FadD3 from P. putida) to γ-decalactone, whereas wildtype YbgC converted 98% 4-hydroxydecanoic acid in this experiment. As can also be seen from Table 12, the mutated YbgC (D18A) enzyme was able only to convert small amounts of 5-hydroxydecanoic acid or 6-hydroxyhexanoic acid to δ-decalactone or ε-caprolactone, whereas wildtype YbgC converted 97% 5-hydroxydecanoic acid or 42% of 6-hydroxyhexanoic acid in this experiment.

These results demonstrated that mutating aspartate 18 to alanine in YbgC from E. coli significantly reduced the ability of the enzyme to catalyze the formation of γ- or δ-decalactone or ε-caprolactone from 4-hydroxydecanoic acid or 5-hydroxydecanoic acid or 6-hydroxyhexanoic acid (via 4-hydroxydecanyl-CoA or 5-hydroxydecanyl-CoA or 6-hydroxyhexanyl-CoA in the presence of FadD3 from P. putida). It also demonstrates that the surprising lactone-forming activity is intrinsic to the ybgC protein and is not an indirect effect resulting from ybgC overexpression.

TABLE 12
Bioconversion to γ- or δ-decalactone or ε-caprolactone
in recombinant E. coli.

| | 4-OH | 5-OH | 6-OH |
|---|---|---|---|
| overexpressed | decanoic acid | decanoic acid | hexanoic acid |
| Gene | Conversion % | Conversion % | Conversion % |
| ybgC | 98% | 97% | 42% |
| none | 0 | 0 | 0 |
| ybgC(D18A) | 30% | 2% | 6% |

As is apparent to one of skill in the art, various modifications and variations of the above aspects and embodiments can be made without departing from the spirit and scope of this disclosure. Such modifications and variations are thus within the scope of this disclosure.

We claim:

1. A method for enzymatically producing a lactone under physiological conditions, the method comprising: culturing a recombinant bacterium that heterologously expresses a YbgC protein and an acyl-CoA synthetase protein, in a culture medium having a neutral pH
wherein
the heterologously expressed YbgC protein converts an hydroxy acyl-CoA substrate to a lactone, and
wherein
the hydroxyl acyl-CoA substrate is a member selected from: a 4-hydroxy acyl-CoA; a 5-hydroxy acyl-CoA and a 6-hydroxy acyl-CoA, and
wherein
the heterologously expressed YbgC protein converts the 4-hydroxy acyl-CoA to a γ-lactone; the heterologously expressed YbgC protein converts the 5-hydroxy acyl-CoA to a δ-lactone and the heterologously expressed YbgC protein converts the 6-hydroxy acyl-CoA, to an ε-lactone.

2. The method of claim 1, wherein the heterologously expressed YbgC protein is overexpressed.

3. The method of claim 1, wherein the hydroxy acyl-CoA substrate is produced by the recombinant bacterium from a fatty acid derivative molecule exogenously added to the culture medium.

4. The method of claim 3, wherein the fatty acid derivative molecule is a 4-hydroxy fatty acid derivative, a 5-hydroxyl fatty acid derivative or a 6-hydroxy fatty acid derivative.

5. The method of claim 4, wherein the fatty acid derivative molecule is a 4-hydroxy fatty acid derivative and the recombinant bacterium produces the 4-hydroxy acyl-CoA from the 4-hydroxy fatty acid derivative.

6. The method of claim 5, wherein the 4-hydroxy fatty acid derivative is 4-hydroxy decanoic acid.

7. The method of claim 3, wherein the recombinant bacterium produces the 5-hydroxy acyl-CoA from the 5-hydroxyl fatty acid derivative.

8. The method of claim 7, wherein the 5-hydroxy fatty acid derivative is 5-hydroxy decanoic acid.

9. The method of claim 3, wherein the recombinant bacterium produces the 6-hydroxy acyl-CoA from the 6-hydroxy fatty acid derivative.

10. The method of claim 9, wherein the 6-hydroxy fatty acid derivative is 6-hydroxy hexanoic acid.

11. The method of claim 1, wherein the heterologously expressed YbgC protein has at least 60% sequence identity to SEQ ID NO: 1 over the full length of the YbgC amino acid sequence.

12. The method of claim 1, wherein the heterologous YbgC protein has at least 70% sequence identity to SEQ ID NO:1 over the full length of the YbgC amino acid sequence.

13. The method of claim 1, wherein the heterologous YbgC protein has at least 75% sequence identity to SEQ ID NO: 1 over the full length of the YbgC amino acid sequence.

14. The method of claim 1, wherein the heterologous YbgC protein has at least 80% sequence identity to SEQ ID NO: 1 over the full length of the YbgC amino acid sequence.

15. The method of claim 1, wherein the heterologous YbgC protein has at least 85% sequence identity to SEQ ID NO: 1 over the full length of the YbgC amino acid sequence.

16. The method of claim 1, wherein the heterologous YbgC protein has at least 90% sequence identity to SEQ ID NO: 1 over the full length of the YbgC amino acid sequence.

17. The method of claim 1, wherein the heterologous YbgC protein has at least 95% sequence identity to SEQ ID NO: 1 over the full length of the YbgC amino acid sequence.

18. The method of claim 1, wherein the heterologous YbgC protein is SEQ ID NO:1.

19. The method of claim 11, wherein the heterologous YbgC protein that has at least 60% sequence identity to SEQ ID NO: 1 over the full length of the YbgC amino acid sequence is a member selected from the group consisting of: Escherichia coli ybgC (SEQ ID NO:1); Citrobacter koseri ybgC (SEQ ID NO:2); Enterobacter cloacae ybgC (SEQ ID NO:3); Serratia fonticola ybgC (SEQ ID NO:4); Exiguobacterium mexicanum ybgC (SEQ ID NO:5) and Plesiomonas shigelloides ybgC (SEQ ID NO: 6).

20. The method of claim 1, wherein the recombinant bacterium further heterologously expresses: a thioesterase and an hydroxylating enzyme.

21. The method of claim 20, wherein the thioesterase is a member selected from SEQ ID NO: 16, SEQ ID NO: 17, SEQ ID NO: 18, SEQ ID NO: 19, SEQ ID NO:20, SEQ ID NO: 21, SEQ ID NO:22, SEQ ID NO:23 and SEQ ID NO:24, and the hydroxylating enzyme is a member selected from SEQ ID NO:25, SEQ ID NO: 26, SEQ ID NO:27, SEQ ID NO:28, SEQ ID NO:29, SEQ ID NO:30 and SEQ ID NO: 31.

22. The method of claim 20, wherein the recombinant bacterium produces the lactone from a simple carbon source.

23. The method of claim 20, wherein the heterologously expressed YbgC protein has at least 60% sequence identity to SEQ ID NO: 1 over the full length of the YbgC amino acid sequence.

24. The method of claim 20, wherein the heterologous YbgC protein has at least 70% sequence identity to SEQ ID NO: 1 over the full length of the YbgC amino acid sequence.

25. The method of claim 20, wherein the heterologous YbgC protein has at least 75% sequence identity to SEQ ID NO: 1 over the full length of the YbgC amino acid sequence.

26. The method of claim 20, wherein the heterologous YbgC protein has at least 80% sequence identity to SEQ ID NO:1 over the full length of the YbgC amino acid sequence.

27. The method of claim 20, wherein the heterologous YbgC protein has at least 85% sequence identity to SEQ ID NO: 1 over the full length of the YbgC amino acid sequence.

28. The method of claim 20, wherein the heterologous YbgC protein has at least 90% sequence identity to SEQ ID NO: 1 over the full length of the YbgC amino acid sequence.

29. The method of claim 20, wherein the heterologous YbgC protein has at least 95% sequence identity to SEQ ID NO: 1 over the full length of the YbgC amino acid sequence.

30. The method of claim 20, wherein the heterologous YbgC protein is SEQ ID NO:1.

31. The method of claim 23, wherein the heterologous YbgC protein that has at least 60% sequence identity to SEQ ID NO: 1 over the full length of the YbgC amino acid sequence is a member selected from the group consisting of: Escherichia coli ybgC (SEQ ID NO:1); Citrobacter koseri ybgC (SEQ ID NO:2); Enterobacter cloacae ybgC (SEQ ID NO:3); Serratia fonticola ybgC (SEQ ID NO:4); Exiguobacterium mexicanum ybgC (SEQ ID NO:5) and Plesiomonas shigelloides ybgC (SEQ ID NO: 6).
